(19) United States
(12) Patent Application Publication
(10) Pub. No.: US 2022/0411495 A1
(43) Pub. Date: Dec. 29, 2022

(54) POSITIVE ALLOSTERIC MODULATORS OF THE CALCIUM-SENSING RECEPTOR

(71) Applicants: VIB VZW, Gent (BE); VRIJE UNIVERSITEIT BRUSSEL, Brussel (BE); UNIVERSITY OF COPENHAGEN, Copenhagen K (DK)

(72) Inventors: Jan Steyaert, Beersel (BE); Els Pardon, Wezemaal (BE); Alexandre Wohlkönig, Brussel (BE); Thomas Zögg, Heverlee (BE); Iris Mos, WB Eersel (NL); Hans Bräuner-Osborne, Copenhagen (DK); Jesper Mosolff Mathiesen, Farum (DK)

(21) Appl. No.: 17/780,068

(22) PCT Filed: Nov. 27, 2020
(86) PCT No.: PCT/EP2020/083749

§ 371 Date: May 26, 2022

(30) Foreign Application Priority Data
Nov. 27, 2019 (EP) 19211709.1

(51) Int. Cl. C07K 16/28; A61P 5/20

(52) U.S. Cl. CPC C07K 16/28; A61P 5/20; C07K 2317/31; C07K 2317/75; C07K 2317/569

(57) ABSTRACT

The present invention relates to non-naturally occurring antibodies or active antibody fragments specifically binding the calcium-sensing receptor (CaSR), acting as positive allosteric modulators (PAMs) to provide for potent therapeutic agents. More particular, the immunoglobulin single variable domains (ISVDs) identified herein reveal a novel therapeutic strategy to reduce parathyroid hormone release in a subject, and are therefore suitable for treatment of hypercalcemia disorders. Moreover, co-application of such an ISVD and a synthetic PAM or calcimimetic results in a synergistic agonistic CaSR activity providing for pharmaceutical compositions as next generation CaSR drugs.

### CROSS-REFERENCE TO RELATED APPLICATIONS

This application is a national phase entry under 35 U.S.C. § 371 of International Patent Application PCT/EP2020/083749, filed Nov. 27, 2020, designating the United States of America and published in English as International Patent Publication WO 2021/105438 on Jun. 3, 2021, which claims the benefit under Article 8 of the Patent Cooperation Treaty to European Patent Application Serial No. 19211709.1, filed Nov. 27, 2019, the entireties of which are hereby incorporated by reference.

### FIELD OF THE INVENTION

The present invention relates to non-naturally occurring antibodies or active antibody fragments specifically binding the calcium-sensing receptor (CaSR), acting as positive allosteric modulators (PAMs) to provide for potent therapeutic agents. More particular, the immunoglobulin single variable domains (ISVDs) identified herein reveal a novel therapeutic strategy to reduce parathyroid hormone release in a subject, and are therefore suitable for treatment of hypercalcemia disorders. Moreover, co-application of such an ISVD and a synthetic PAM or calcimimetic results in a synergistic agonistic CaSR activity providing for pharmaceutical compositions as next generation CaSR drugs.

### BACKGROUND

The calcium-sensing receptor (CaSR) is responsible for maintaining a constant stable serum calcium concentration in an organism, such as in the human body (Brown et al., 1993; Brown, 2013). Accordingly, this class C G protein-coupled receptor (GPCR) is highly expressed in calciotropic tissues (i.e. parathyroid gland, bones and kidney) and associated with several calcium homeostasis disorders that are either from genetic or acquired origin (Vahe et al., 2017; Hannan et al., 2018). CaSR is predominantly expressed on the cell-surface as a homodimer where it can be activated by numerous natural ligands and positively modulated by proteinogenic L-amino acids (Bai et al., 1998; Hofer and Brown, 2003). Consequently, CaSR is a well-established drug target in calcium homeostasis disorders. Synthetic ligands modulating CaSR activity have proven to be of therapeutic importance. Cinacalcet (Sensipar®) is an approved small synthetic drug that acts as a positive allosteric modulator (PAM) of CaSR by binding to the CaSR seven transmembrane domain, and used as an oral treatment for hypercalcemia in adult patients with parathyroid carcinomas, for primary hyperparathyroidism in patients who cannot undergo parathyroidectomy and for secondary hyperparathyroidism in chronic kidney disease patients receiving hemodialysis (Poon, 2005). Unfortunately, its clinical use is limited due to the high risk of hypocalcemia and gastro-intestinal intolerance (Bover et al., 2016). Alternative PAMs include a peptide ligand etelcalcetide (Parsabiv®) and a small molecule evocalcet (Orkedia®) approved for the treatment of secondary hyperparathyroidism in chronic kidney disease patients receiving hemodialysis. Treatment with currently approved CaSR therapeutics is unfortunately restricted by systemic adverse effects (Hamano et al., 2017; Fukagawa et al., 2018; Kawata et al., 2018; Patel and Bridgeman, 2018).

Expression of CaSR extends to other tissues beyond the extracellular Ca²⁺ homeostatic system including the cardiovascular system, the airways, and the nervous system where it may play physiological functions. So, CaSR is involved in different pathologies including uncontrolled blood pressure, vascular calcification, asthma, and Alzheimer's disease. Finally, the CaSR has been shown to play a critical role in cancer either contributing to bone metastasis and/or acting as a tumor suppressor in some forms of cancer (parathyroid cancer, colon cancer, and neuroblastoma) and as oncogene in others (breast and prostate cancers). Negative allosteric modulators (NAMs) or calcilytic drugs, which block CaSR activity, have been investigated for increased bone density in animals for the treatment of osteoporosis.

GPCR drug discovery has expanded from synthetic molecules and peptides towards the generation of GPCR-targeting antibodies or antibody fragments. In contrast to synthetic drugs, antibodies generally show high target specificity which lowers their off-target effects and offer the opportunity to adjust their in vivo half-life and/or to tissue specificity via nanobody engineering (Bates and Power, 2019). A nanobody or VHH fragment is a stable single-domain fragment of a heavy chain only antibody that naturally occurs in sera from camelids or cartilaginous fish (Hamers-Casterman C et al., 1993; Greenberg et al., 1995; Dooley and Flajnik, 2006). The small size (˜15 kDa) makes the nanobody more stable, easier to produce and modify, and easier to handle than regular antibodies (Bates and Power, 2019). With the recent first FDA approval of a nanobody (caplacizumab) for the treatment of acquired thrombotic thrombocytopenic purpura, the therapeutic potential of nanobodies has been established (Morrison, 2019). Conversely, human autoantibodies recognizing the calcium-sensing receptor have been found in several patient populations (e.g. Li et al., 1996; Habibullah et al., 2018). These antibodies are shown to bind the extracellular domain (ECD) of the CaSR protein, still they differ in their pharmacological profiles as compared to antibody-based therapeutics. Furthermore, Yang et al. (WO2017/172944) describe the crystal structure of the ECD of CaSR in complex with tool compounds, and disclosed antibodies and fragments thereof binding the ECD of CaSR. However, as opposed to the known synthetic PAMs, no ECD-specific CaSR antibodies or antibody fragments inducing an allosteric effect, or in particular a PAM effect on CaSR are known to date. It would be interesting to identify CaSR-specific antibody-based agents binding to the extracellular part of the receptor and which provide for a positive allosteric effect in the presence of calcium provide, thereby creating the potential for a new generation of or tools for exploring improved therapeutic drug discovery to evolve into a more precise and effective treatment of CaSR-related disorders.

### SUMMARY OF THE INVENTION

The present invention discloses eight pharmacologically potent calcium-sensing receptor (CaSR)-specific nanobodies, classified in five different nanobody families, with positive allosteric modulation (PAM) activity for the CaSR protein. These CaSR-specific nanobodies were shown to act as positive allosteric modulators (PAMs) in Ca²⁺-mediated CaSR activation, and therefore form a new class of pharmacological calcimimetic biological compounds, which potentially can be developed into new therapeutics in the treatment of CaSR-related disorders.

In a first aspect, the invention relates to a protein binding agent specifically binding the CaSR, wherein the protein binding agent comprises an antibody or active antibody fragment, as defined herein, and which constitutes positive allosteric modulating activity upon binding to CaSR. Said protein binding agents comprising an antibody or active antibody fragment are different from human or naturally-occurring autoantibodies for CaSR, which constitute IgG1 antibodies, a conventional type not in the scope of this invention. In another embodiment, the protein binding agent comprising an antibody or active antibody fragment specifically binds the CaSR protein when added extracellularly to intact CaSR-expressing cells. Alternatively, said antibody or active antibody fragment specifically binds the extracellular calcium binding domain (ECD) and/or the extracellularly accessible portion of the seven-transmembrane domain of CaSR. In another embodiment, the protein binding agents disclosed herein comprise an antibody or active antibody fragment and further comprise peptides, peptidomimetics, antibody mimetics, single domain antibodies, or immunoglobulin single variable domains (ISVDs). In the particular embodiment said protein binding agent comprises an antibody or active antibody fragment comprising at least one or more ISVDs, said ISVDs comprise 4 framework regions (FR) and 3 complementarity determining regions (CDR) according to the following formula (1): FR1-CDR1-FR2-CDR2-FR3-CDR3-FR4 (1). More specifically, said ISVDs as disclosed herein may comprise a CDR3 region comprising a sequence selected from SEQ ID NOs:24-30. A further specific embodiment relates to said ISVDs with said CDR3 sequence selected from SEQ ID NO:24-30, and may further comprise a CDR1 consisting of a sequence selected from the group of SEQ ID NO: 11-16, and CDR2 consisting of a sequence selected from the group of SEQ ID NO: 17-23. A specific embodiment relates to said ISVDs comprising a sequence for CDR1, 2 and 3, which may be provided as an ISVD with CDR1 being SEQ ID NO:11, CDR2 as SEQ ID NO:17 and CDR as SEQ ID NO:24; or CDR1 as SEQ ID NO:12, CDR2 as SEQ ID NO:18 and CDR3 as SEQ ID NO: 25; or as an ISVD with CDR1 as SEQ ID NO:12, CDR2 as SEQ ID NO:19 and CDR3 as SEQ ID NO: 26; or as an ISVD with CDR1 as SEQ ID NO:13, CDR2 as SEQ ID NO:20 and CDR3 as SEQ ID NO: 27; or as an ISVD with CDR1 as SEQ ID NO:14, CDR2 as SEQ ID NO:21 and CDR3 as SEQ ID NO: 28; or as an ISVD with CDR1 as SEQ ID NO: 15, CDR2 as in SEQ ID NO: 22 and CDR3 as in SEQ ID NO: 29; or an ISVD with CDR1 as SEQ ID NO:16, CDR2 as SEQ ID NO:23 and CDR3 as SEQ ID NO: 30.

A specific embodiment relates to said ISVD comprising a sequence selected from the group of sequences of SEQ ID NO: 3-10, or a sequence with at least 85% amino acid identity thereof, or a humanized variant thereof. A further specific embodiment relates to said protein binding agents of the invention, acting as CaSR-binding PAMs, comprising an antibody or active antibody fragment comprising one or more ISVDs which are humanized variants of any of the sequences of the Nbs of SEQ ID NO:3-10, even more specifically humanized variants of Nb36, Nb4, Nb2 or Nb10, for instance humanized variants as depicted in SEQ ID NO:31-34. Another embodiment of the present invention discloses a multi-specific or multivalent protein binding agent, which comprises said protein binding agent as disclosed herein, comprising an antibody or active antibody fragment with PAM activity, and a further protein binding agent with a different or same target specificity, respectively.

A further embodiment provides for a pharmaceutical composition comprising the protein binding agent, such as the antibody or active antibody fragment as disclosed herein, or the multispecific or multivalent protein binding agent as disclosed herein. Alternatively, a pharmaceutical composition is provided for comprising said protein binding agent as disclosed herein, acting as a PAM, as well as a synthetic compound known to have PAM CaSR activity. More specifically, said pharmaceutical composition may contain a mixture of several binding agents wherein said mixture comprises at least one small molecule CaSR PAM, and at least one antibody or active antibody fragment CaSR-specific PAM or multivalent or multi-specific binding agent comprising said antibody or active antibody fragment as described herein. Said at least one antibody or active antibody fragment may be specifically present in said pharmaceutical composition with CDR sequences selected from the CDR1, 2 and 3 sequences of Nb10, Nb4 and Nb2, i.e. may be provided as an ISVD with CDR1 being SEQ ID NO:11, CDR2 as SEQ ID NO:17 and CDR as SEQ ID NO:24; or CDR1 as SEQ ID NO:12, CDR2 as SEQ ID NO:18 and CDR3 as SEQ ID NO: 25; or as an ISVD with CDR1 as depicted in SEQ ID NO: 15, CDR2 as in SEQ ID NO: 22 and CDR3 as in SEQ ID NO: 29.

Another aspect of the invention relates to the protein binding agent as disclosed herein, or the pharmaceutical compositions provided for, for use as a medicament. Specific embodiments relate to the protein binding agent or the pharmaceutical compositions as described herein, for use as in treatment of a subject to reduce parathyroid hormone secretion.

Another embodiment so relates to the antibody or active antibody fragment protein binding agents or the pharmaceutical compositions as described herein, for use in treatment of CaSR-related disorders. Said disorders may be hypercalcemia disorders, such as secondary and primary hyperparathyroidism, but may also involve disorders with vascular calcification, asthma, or related to Alzheimer disease. Furthermore, said disorders may be caused by loss-of-function mutations in the CaSR (familial hypocalciuric hypercalcemia type 1 (FHH1) or neonatal severe hyperparathyroidism (NSHPT)), Gα₁₁ protein (FHH2) or heterotetrameric adaptor-related protein complex-2 (AP2σ, FHH3).

A final aspect relates to a protein complex comprising the CaSR extracellular calcium binding domain, and a protein binding agent as disclosed herein, wherein the protein binding agent is bound to the CaSR receptor protein extracellular part. In a specific embodiment, said complex is crystalline. Finally, said complex may be used to define the 3-dimensional structure, possibly using cryo-electron microscopy.

### DESCRIPTION OF THE FIGURES

The patent or application file contains at least one drawing executed in color. Copies of this patent or patent application publication with color drawing(s) will be provided by the Office upon request and payment of the necessary fee.

The drawings described are only schematic and are non-limiting. In the drawings, the size of some of the elements may be exaggerated and not drawn on scale for illustrative purposes.

FIG. 1. Representative SDS-PAGE Gel Analysis of Purified Nanobodies.

Samples were analysed on a 4-20% precast polyacrylamide gel followed by Coomassie blue staining. Lane 1, pre-stained protein marker (M). Lanes 2-10, IMAC column flow-through before washing (FT), column flow-through after washing with 4×8 mL buffer containing 10 mM imidazole (W) and nanobody elution after addition of 3×700 μL buffer containing 500 mM imidazole (E) of three arbitrary nanobodies. Lanes 11-15, IMAC nanobody elution further purified by size exclusion chromatography (SEC) of 5 arbitrary nanobodies.

FIGS. 2A-2C. Pharmacological Characterization of a CaSR Nanobody Library in a Flp-In HEK293 Cell Line Recombinantly Expressed with Human CaSR Wild Type (WT).

(FIGS. 2A and 2B) Screening of a purified nanobody library for pharmacological activity at EC₂₀ Ca²⁺ in (FIG. 2A) the IP1 accumulation assay and (FIG. 2B) the phospho-ERK1/2 assay. (FIG. 2C) IP₁ accumulation of hCaSR WT upon stimulation with EC₂₀ Ca²⁺ and increasing pharmacologically active nanobody or calcimimetic NPS R-568 concentrations. Data in (FIGS. 2A and 2B) are presented as mean±S.D. of a single experiment performed in duplicate, while data in (FIG. 2C) are mean±S.E.M. of three independent experiments performed in triplicate.

FIGS. 3A-3D. Studying Synergism of CaSR Nanobody/Nanobody and CaSR Nanobody/NPS R-568 Combinations in the IP-One Accumulation Assay.

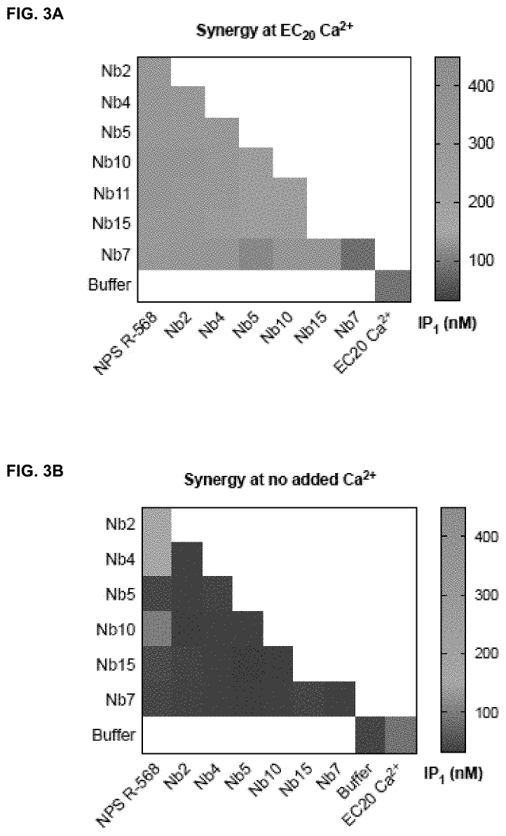

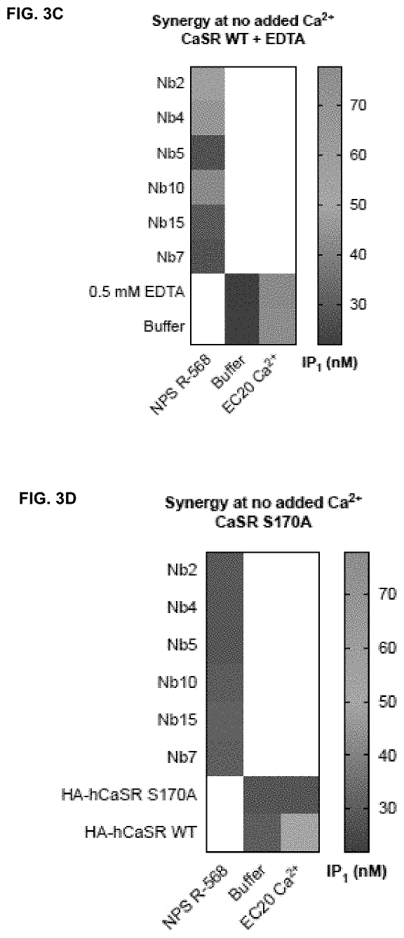

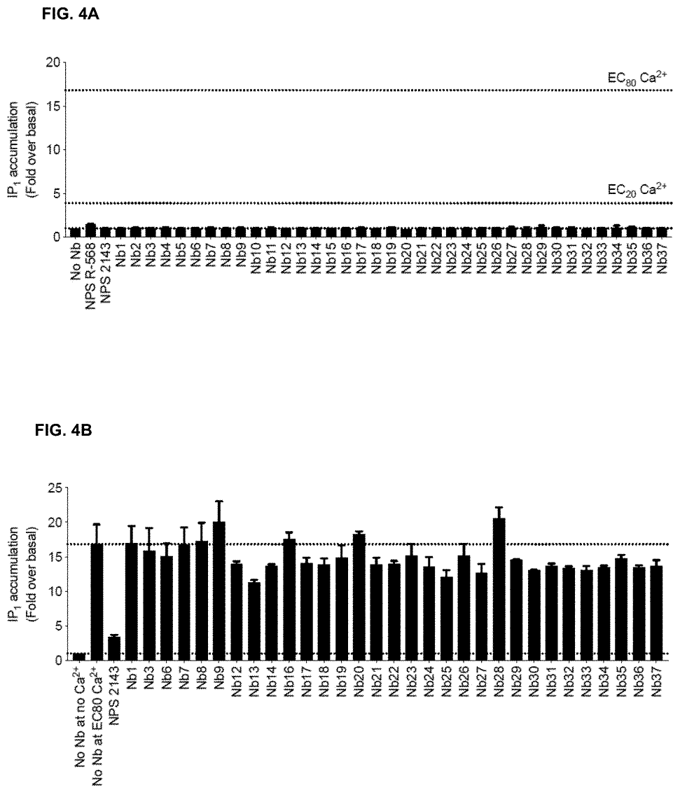

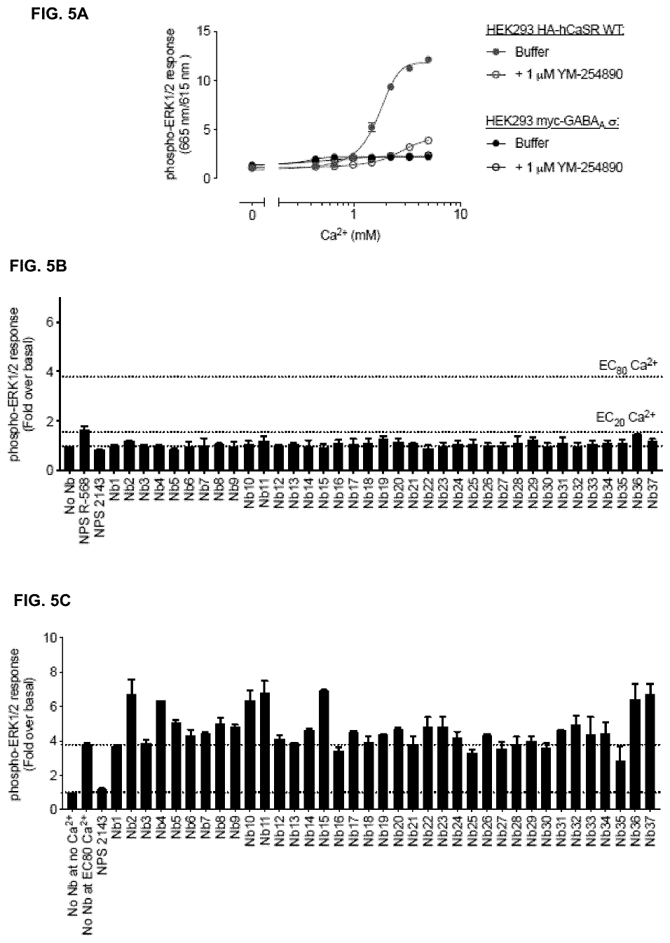

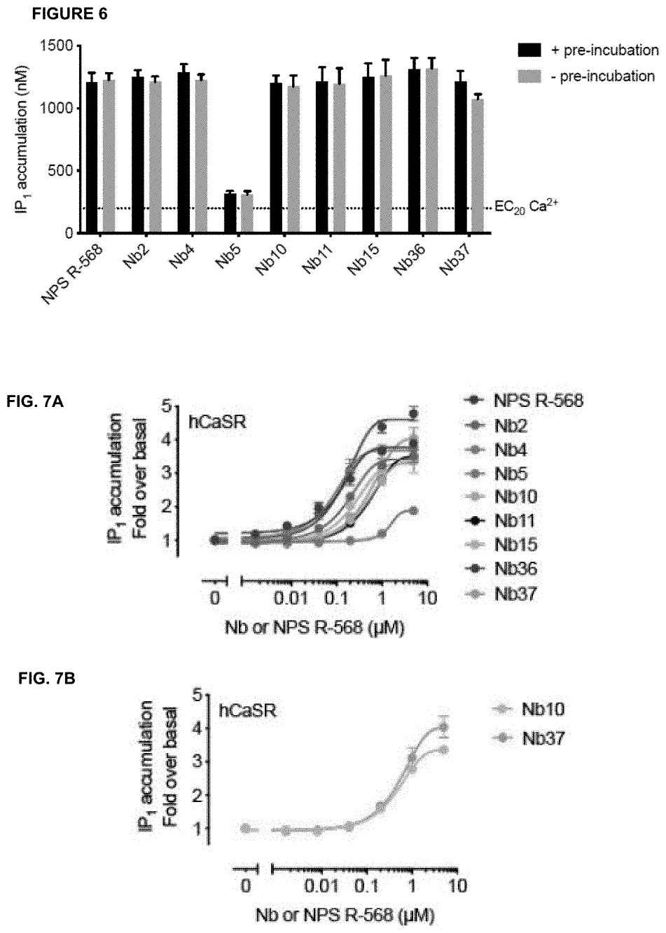

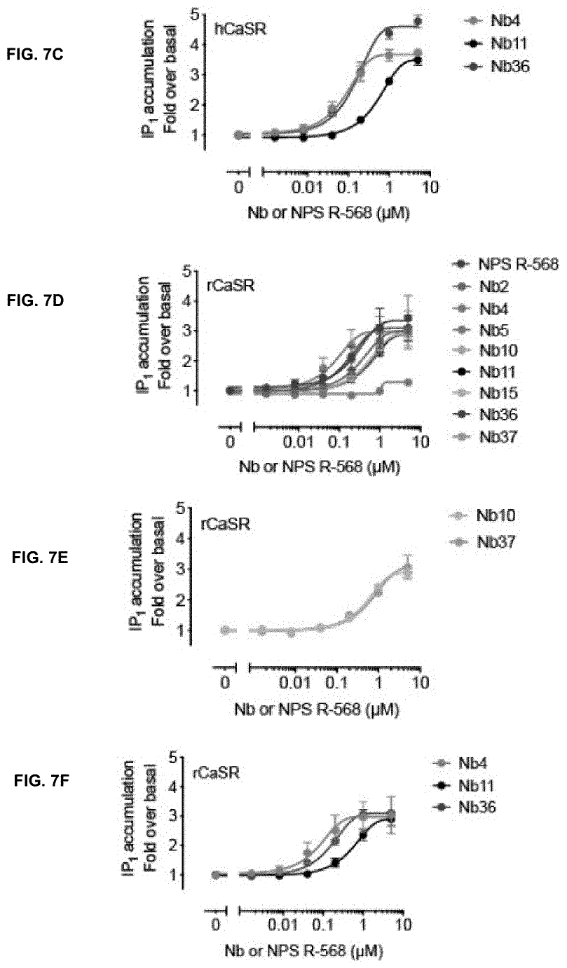

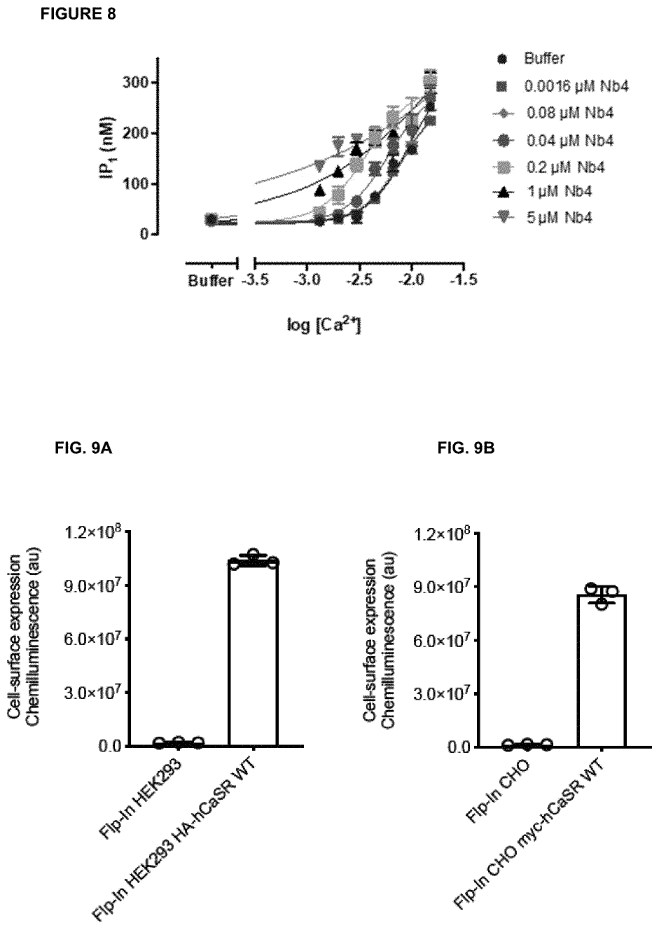

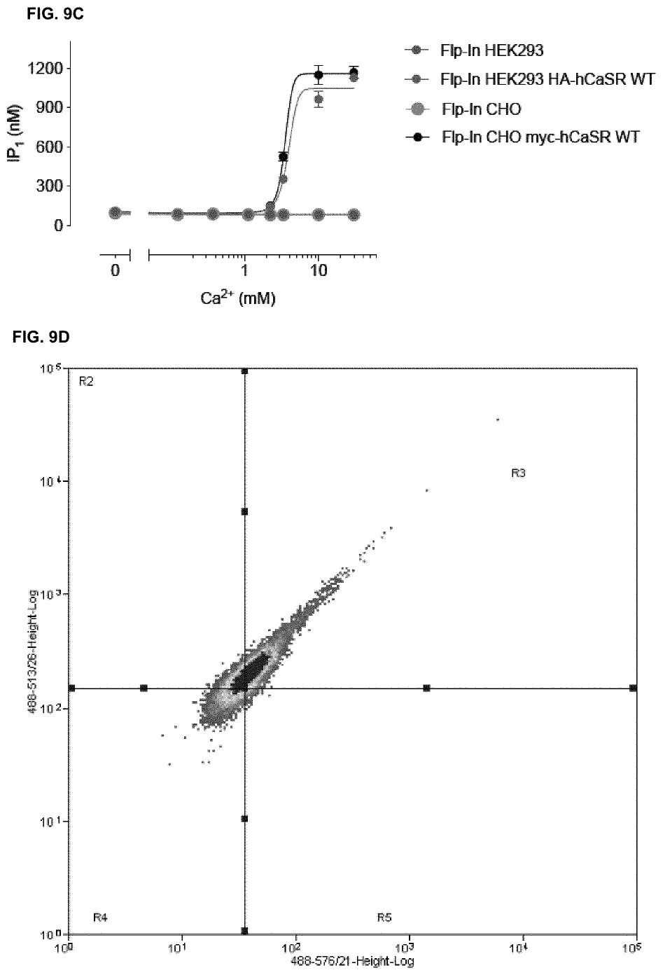

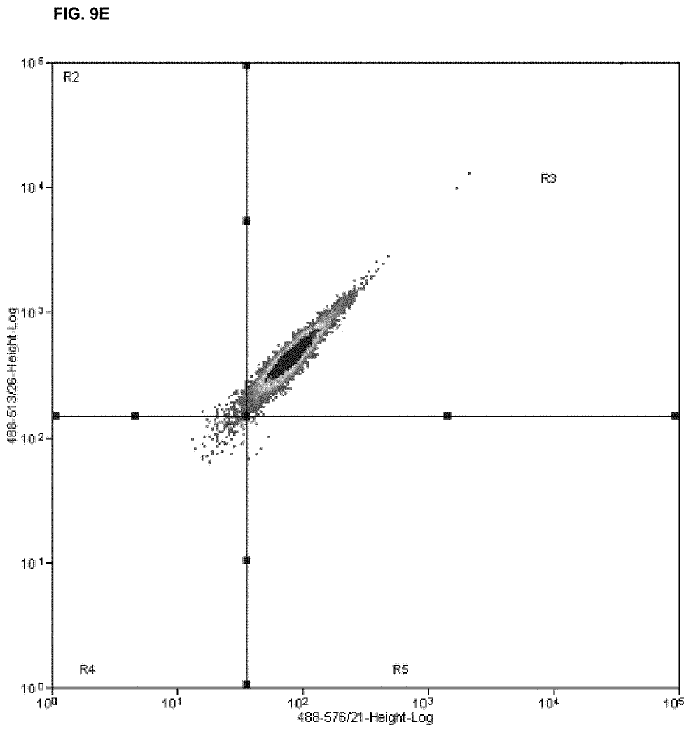

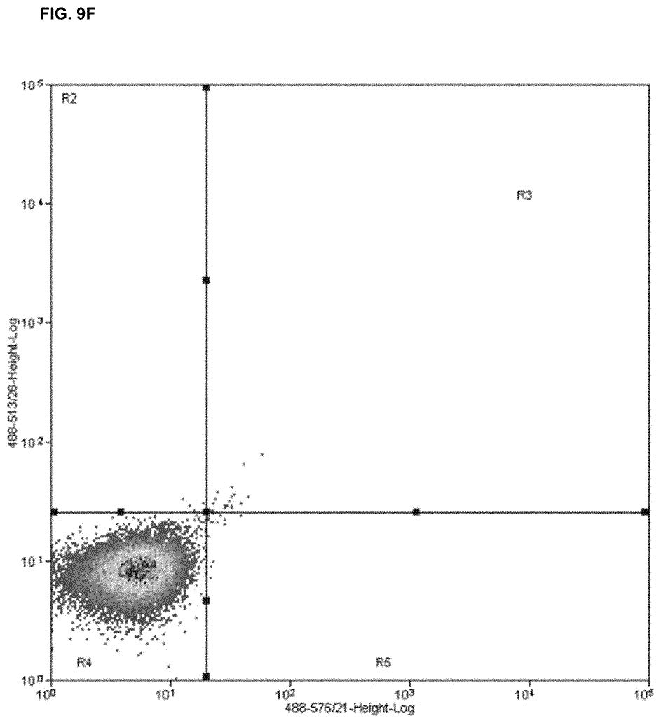

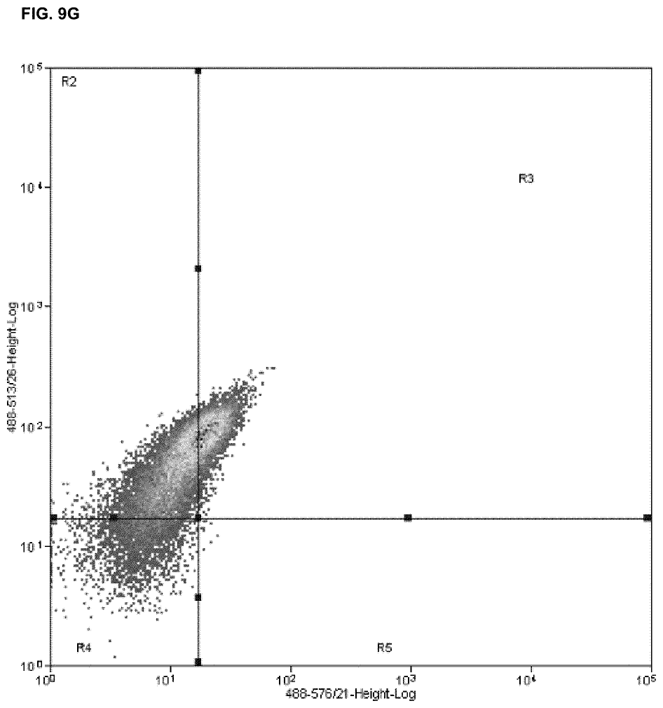

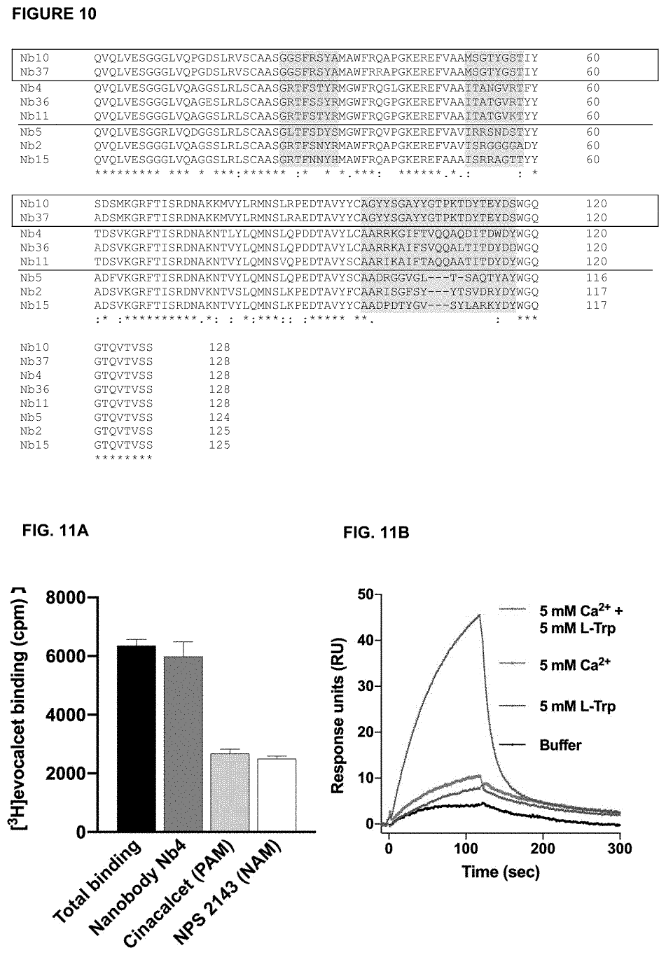

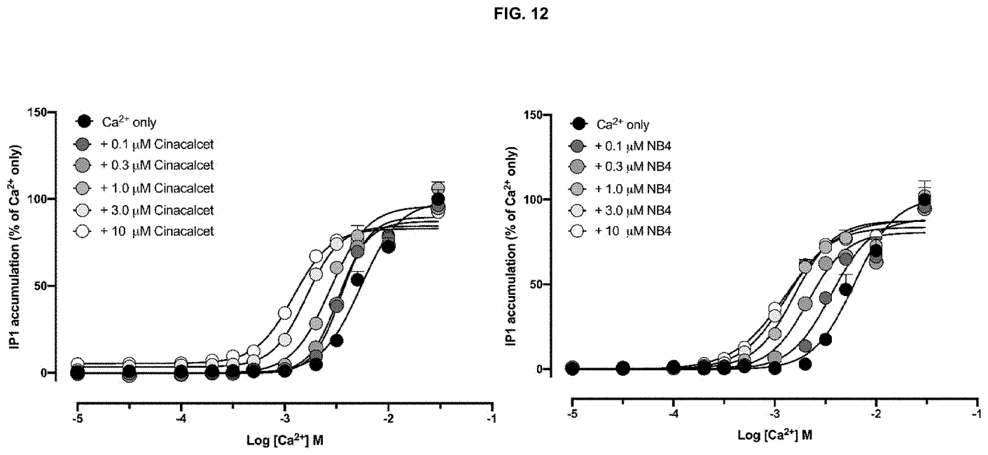

HEK293 HA-hCaSR WT cells were stimulated with different nanobody/nanobody or NPS R-568/nanobody combinations in a 1:1 ratio and a total concentration of 10 μM. Combinations with the inactive nanobody Nb7 correspond to the active nanobody or NPS R-568 response alone. (FIGS. 3A and 3B) Synergy studies were performed in the (FIG. 3A) presence or (FIG. 3B) absence of EC₂₀ Ca²⁺. (FIGS. 3C and 3D) Synergy studies of different nanobody/NPS R-568 combinations in the absence of added Ca²⁺ and (FIG. 3A) 0.5 mM EDTA or (FIG. 3B) loss-of-function HA-hCaSR S170A mutant. IP₁ accumulation concentrations are displayed as colours ranging from red to green. Individual squares in the plotted heatmap represent the mean IP₁ accumulation concentration of three independent experiments performed in triplicate.

FIGS. 4A and 4B. Pharmacological Screen for Nanobodies with PAM or NAM Activity in the IP-One Accumulation Assay.

A fixed nanobody concentration (5 μM) was tested in the IP-One accumulation assay in (FIG. 4A) the absence of Ca²⁺ or (FIG. 4B) presence of EC₈₀ Ca²⁺ in an attempt to find pharmacologically active nanobodies acting as PAM or NAM, respectively. Data are mean±S.D. of a single experiment performed in duplicate.

FIGS. 5A-5C. Pharmacological Screen for CaSR Nanobodies with Agonist or NAM Activity in the G_{q/11}-Dependent Phospho-ERK1/2 Assay.

(FIG. 5A) Ca²⁺-mediated ERK1/2 phosphorylation measured in stable HEK293 HA-hCaSR WT (red) and HEK293 myc-GABA (black) cells in the absence (closed symbols) or presence (open symbols) of 1 μM G_{q/11} inhibitor YM-254890. (FIGS. 5B and 5C) A fixed nanobody concentration (5 μM) was tested in the phospho-ERK1/2 assay in (FIG. 5B) the absence of Ca²⁺ or (FIG. 5C) presence of EC₈₀ Ca²⁺ in an attempt to find pharmacologically active nanobodies acting as agonist or NAM, respectively. Data in (FIG. 5A) are mean±S.E.M. of three experiments performed in triplicate and data in (FIGS. 5B and 5C) are mean±S.D. of a single experiment performed in duplicate.

FIG. 6. Nanobody and NPS R-568 Pre-Incubation Test.

IP₁ accumulation response obtained for 5 μM NPS R-568 or pharmacologically active nanobodies with (+) or without (−) 30 minutes pre-incubation at 37° C. prior to stimulation with EC₂₀ Ca²⁺. The dotted line indicates the IP₁ response at EC₂₀ Ca²⁺ only. Data are mean±S.E.M. of three independent experiments performed in triplicate.

FIGS. 7A-7F. Nanobody and NPS R-568 Concentration-Response Curves at EC₂₀ Ca²⁺ for the CaSR Human and Rat Ortholog.

IP₁ accumulation of (FIGS. 7A-7C) human (hCaSR) and (FIGS. 7D-7F) rat CaSR (rCaSR) upon stimulation with EC₂₀ Ca²⁺ and increasing nanobody or NPS R-568 concentrations. (FIG. 7A and FIG. 7D) overview of all tested nanobodies together and NPS R-568, (FIG. 7B and FIG. 7E) Nb10 and Nb37 alone and (FIG. 7C and FIG. 7F) Nb4, Nb11 and Nb36 alone. Data are presented as mean±S.E.M. of three independent experiments performed in triplicate.

FIG. 8. The Effect of Increasing Nb4 Concentrations on the Loss-of-Function Mutant CaSR Q459R Ca²⁺ Concentration-Response Curve.

FIGS. 9A-9F. Cell Line Validation of Polyclonal Stable Cell Lines Flp-In HEK293 HA-hCaSR WT and Flp-In CHO Myc-hCaSR WT.

Confirmation of CaSR cell-surface expression in (FIG. 9A) Flp-In HEK293 HA-hCaSR WT and (FIG. 9B) Flp-In CHO myc-hCaSR WT. (FIG. 9C) Concentration-dependent IP₁ accumulation in response to Ca²⁺. (FIGS. 9D-9G) Flow cytometry 2D fluorescence plots obtained with secondary antibody Alexa Fluor 488 goat anti-mouse IgG and (FIGS. 9D and 9E) primary mouse anti-HA antibody or (FIGS. 9F and 9G) primary mouse anti-myc antibody. The parental cell lines were included as controls. Data in (FIGS. 9A-9C) are means±S.D. of a single experiment performed in triplicate.

FIG. 10. Protein Alignment of Eight PAM Nanobody Sequences.

The multiple sequence alignment as shown herein for 8 Nbs belonging to 5 families: the first family with Nb10 (SEQ ID NO:3) and Nb37 (SEQ ID NO:4); the second one with Nb4 (SEQ ID NO:5), Nb 36 (SEQ ID NO:7), and Nb11 (SEQ ID NO:6); and the further families with Nb2 (SEQ ID NO:9), Nb15 (SEQ ID NO:10), and Nb5 (SEQ ID NO:8). The CDRs are labelled in grey boxes for CDR1, CDR2 and CDR3, respectively, and in that order, annotated according to IMGT nomenclature.

FIGS. 11A and 11B. Nb4 Binding to the ECD of CaSR.

FIG. 11A) Small-molecule allosteric modulators cinacalcet and NPS 2143 but not nanobody Nb4 displace [3H]evocalcet binding. FIG. 11B) Endogenous CaSR ligands increase Nb4 binding to the extracellular Venus flytrap domain (added at time 0 and washed away at 120 sec, measured by surface plasmon resonance).

FIG. 12. Dose-Dependent Effect of Nb4 Acting as PAM.

IP₁ accumulation of human (hCaSR) upon stimulation with increasing Ca²⁺ concentrations in the presence of varying concentrations of cinacalcet (left) or nanobody Nb4 (right). Increasing concentrations of either cinacalcet or nanobody Nb4 potentiate the Ca²⁺ response and lead to a leftward shift of the concentration-response curves. Data were normalized to the maximal Ca²⁺ response in the absence of PAM and are presented as mean±S.D. of a representative experiment performed in duplicate.

### DETAILED DESCRIPTION

The present invention will be described with respect to particular embodiments and with reference to certain drawings but the invention is not limited thereto but only by the claims. Any reference signs in the claims shall not be construed as limiting the scope. Of course, it is to be understood that not necessarily all aspects or advantages may be achieved in accordance with any particular embodiment of the invention. Thus, for example those skilled in the art will recognize that the invention may be embodied or carried out in a manner that achieves or optimizes one advantage or group of advantages as taught herein without necessarily achieving other aspects or advantages as may be taught or suggested herein. The invention, both as to organization and method of operation, together with features and advantages thereof, may best be understood by reference to the following detailed description when read in conjunction with the accompanying drawings. The aspects and advantages of the invention will be apparent from and elucidated with reference to the embodiment(s) described hereinafter. Reference throughout this specification to “one embodiment” or “an embodiment” means that a particular feature, structure or characteristic described in connection with the embodiment is included in at least one embodiment of the present invention. Thus, appearances of the phrases ‘in one embodiment’ or ‘in an embodiment’ in various places throughout this specification are not necessarily all referring to the same embodiment but may.

### Definitions

Where an indefinite or definite article is used when referring to a singular noun e.g. “a” or “an”, “the”, this includes a plural of that noun unless something else is specifically stated. Where the term “comprising” is used in the present description and claims, it does not exclude other elements or steps.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments, of the invention described herein are capable of operation in other sequences than described or illustrated herein. The following terms or definitions are provided solely to aid in the understanding of the invention. Unless specifically defined herein, all terms used herein have the same meaning as they would to one skilled in the art of the present invention.

Practitioners are particularly directed to Sambrook et al., Molecular Cloning: A Laboratory Manual, 4^{th} ed., Cold Spring Harbor Press, Plainsview, N.Y. (2012); and Ausubel et al., Current Protocols in Molecular Biology (Supplement 114), John Wiley & Sons, New York (2016), for definitions and terms of the art. Unless defined otherwise, all technical and scientific terms used herein have the same meaning as commonly understood by one of ordinary skill in the art (e.g. in molecular biology, biochemistry, structural biology, and/or computational biology).

The terms “protein”, “polypeptide”, and “peptide” are interchangeably used further herein to refer to a polymer of amino acid residues and to variants and synthetic analogues of the same. A “peptide” may also be referred to as a partial amino acid sequence derived from its original protein, for instance after tryptic digestion. Thus, these terms apply to amino acid polymers in which one or more amino acid residues is a synthetic non-naturally occurring amino acid, such as a chemical analogue of a corresponding naturally occurring amino acid, as well as to naturally-occurring amino acid polymers. This term also includes posttranslational modifications of the polypeptide, such as glycosylation, phosphorylation and acetylation. Based on the amino acid sequence and the modifications, the atomic or molecular mass or weight of a polypeptide is expressed in (kilo)dalton (kDa). By “isolated” or “purified” is meant material that is substantially or essentially free from components that normally accompany it in its native state. For example, an “isolated polypeptide” or “purified polypeptide” refers to a polypeptide which has been purified from the molecules which flank it in a naturally-occurring state, e.g., an antibody or nanobody as identified and disclosed herein which has been removed from the molecules present in the sample or mixture, such as a production host, that are adjacent to said polypeptide. An isolated protein or peptide can be generated by amino acid chemical synthesis or can be generated by recombinant production or by purification from a complex sample.

“Homologue”, “Homologues” of a protein encompass peptides, oligopeptides, polypeptides, proteins and enzymes having amino acid substitutions, deletions and/or insertions relative to the unmodified protein in question and having similar biological and functional activity as the unmodified protein from which they are derived. The term “amino acid identity” as used herein refers to the extent that sequences are identical on an amino acid-by-amino acid basis over a window of comparison. Thus, a “percentage of sequence identity” is calculated by comparing two optimally aligned sequences over the window of comparison, determining the number of positions at which the identical amino acid residue (e.g., Ala, Pro, Ser, Thr, Gly, Val, Leu, lie, Phe, Tyr, Trp, Lys, Arg, His, Asp, Glu, Asn, Gln, Cys and Met, also indicated in one-letter code herein) occurs in both sequences to yield the number of matched positions, dividing the number of matched positions by the total number of positions in the window of comparison (i.e., the window size), and multiplying the result by 100 to yield the percentage of sequence identity. A “substitution”, or “mutation”, or “variant” as used herein, results from the replacement of one or more amino acids or nucleotides by different amino acids or nucleotides, respectively as compared to an amino acid sequence or nucleotide sequence of a parental protein or a fragment thereof. It is understood that a protein or a fragment thereof may have conservative amino acid substitutions which have substantially no effect on the protein's activity.

“Binding” means any interaction, be it direct or indirect. A direct interaction implies a contact between the binding partners. An indirect interaction means any interaction whereby the interaction partners interact in a complex of more than two molecules. The interaction can be completely indirect, with the help of one or more bridging molecules, or partly indirect, where there is still a direct contact between the partners, which is stabilized by the additional interaction of one or more molecules. By the term “specifically binds,” as used herein is meant a binding domain which recognizes a specific target, but does not substantially recognize or bind other molecules in a sample. Specific binding does not mean exclusive binding. However, specific binding does mean that proteins have a certain increased affinity or preference for one or a few of their binders. The term “affinity”, as used herein, generally refers to the degree to which a ligand, chemical, protein or peptide binds to another (target) protein or peptide so as to shift the equilibrium of single protein monomers toward the presence of a complex formed by their binding. As used herein, the term “protein complex” or “complex” or “assembled protein(s)” refers to a group of two or more associated macromolecules, whereby at least one of the macromolecules is a protein. A protein complex, as used herein, typically refers to associations of macromolecules that can be formed under physiological conditions. Individual members of a protein complex are linked by non-covalent interactions. A protein complex can be a non-covalent interaction of only proteins, and is then referred to as a protein-protein complex; for instance, a non-covalent interaction of two proteins, of three proteins, of four proteins, etc. More specifically, a complex of the protein binding agent and the CaSR protein, optionally with a ligand bound to it, such as calcium.

A “binding agent” relates to a molecule that is capable of binding to another molecule, wherein said binding is preferably a specific binding, recognizing a defined binding site, pocket or epitope. The binding agent may be of any nature or type and is not dependent on its origin. The binding agent may be chemically synthesized, naturally occurring, recombinantly produced (and purified), as well as designed and synthetically produced. Said binding agent may hence be a small molecule, a chemical, a peptide, a polypeptide, an antibody, or any derivatives thereof, such as a peptidomimetic, an antibody mimetic, an active fragment, a chemical derivative, among others. The term “binding pocket” or “binding site” refers to a region of a molecule or molecular complex, that, as a result of its shape and charge, favourably associates with another chemical entity, compound, proteins, peptide, antibody or Nb. The term “pocket” includes, but is not limited to cleft, channel or site. The term “part of a binding pocket/site” refers to less than all of the amino acid residues that define the binding pocket, or binding site. For example, the portion of residues may be key residues that play a role in ligand binding, or may be residues that are spatially related and define a three-dimensional compartment of the binding pocket. The residues may be contiguous or non-contiguous in primary sequence. For antibody-related molecules, the term “epitope” is also used to describe the binding site, as used interchangeably herein.

An “epitope”, refers to an antigenic determinant of a polypeptide, constituting a binding site or binding pocket on a target molecule, such as the CaSR protein, more specifically a binding pocket on the CaSR extracellular calcium-binding domain (ECD) or transmembrane domain, which is an accessible epitope or binding site on the extracellular side of the cell. An epitope could comprise 3 amino acids in a spatial conformation, which is unique to the epitope. Generally, an epitope consists of at least 4, 5, 6, 7 such amino acids, and more usually, consists of at least 8, 9, 10 such amino acids. Methods of determining the spatial conformation of amino acids are known in the art, and include, for example, X-ray crystallography and multi-dimensional nuclear magnetic resonance. A “conformational epitope”, as used herein, refers to an epitope comprising amino acids in a spatial conformation that is unique to a folded 3-dimensional conformation of a polypeptide. Generally, a conformational epitope consists of amino acids that are discontinuous in the linear sequence but that come together in the folded structure of the protein. However, a conformational epitope may also consist of a linear sequence of amino acids that adopts a conformation that is unique to a folded 3-dimensional conformation of the polypeptide (and not present in a denatured state). In protein complexes, conformational epitopes consist of amino acids that are discontinuous in the linear sequences of one or more polypeptides that come together upon folding of the different folded polypeptides and their association in a unique quaternary structure. Similarly, conformational epitopes may here also consist of a linear sequence of amino acids of one or more polypeptides that come together and adopt a conformation that is unique to the quaternary structure. The term “conformation” or “conformational state” of a protein refers generally to the range of structures that a protein may adopt at any instant in time. One of skill in the art will recognize that determinants of conformation or conformational state include a protein's primary structure as reflected in a protein's amino acid sequence (including modified amino acids) and the environment surrounding the protein. The conformation or conformational state of a protein also relates to structural features such as protein secondary structures (e.g., α-helix, β-sheet, among others), tertiary structure (e.g., the three dimensional folding of a polypeptide chain), and quaternary structure (e.g., interactions of a polypeptide chain with other protein subunits). Posttranslational and other modifications to a polypeptide chain such as ligand binding, phosphorylation, sulfation, glycosylation, or attachments of hydrophobic groups, among others, can influence the conformation of a protein. Furthermore, environmental factors, such as pH, salt concentration, ionic strength, and osmolality of the surrounding solution, and interaction with other proteins and co-factors, among others, can affect protein conformation. The conformational state of a protein may be determined by either functional assay for activity or binding to another molecule or by means of physical methods such as X-ray crystallography, NMR, or spin labeling, among other methods. For a general discussion of protein conformation and conformational states, one is referred to Cantor and Schimmel, Biophysical Chemistry, Part I: The Conformation of Biological. Macromolecules, W.H. Freeman and Company, 1980, and Creighton, Proteins: Structures and Molecular Properties, W.H. Freeman and Company, 1993.

The term “antibody”, “antibody fragment” and “active antibody fragment” as used herein refer to a protein comprising at least an immunoglobulin (Ig) domain or an antigen binding domain capable of specifically binding the antigen, in this case the CaSR protein. ‘Antibodies’ can further be intact immunoglobulins derived from natural sources or from recombinant sources and can be immunoreactive portions of intact immunoglobulins. Antibodies are typically tetramers of immunoglobulin molecules.

The term “active antibody fragment” refers to a portion of any antibody or antibody-like structure that by itself has high affinity for an antigenic determinant, or epitope, and contains one or more CDRs accounting for such specificity. Preferably an ‘active antibody fragment’ is not a conventional antibody and only constitutes the Variable domain(s), lacking the constant domains (as present in conventional antibodies). Non-limiting examples include immunoglobulin domains, which may be derived from any type of antibody, or Fabs, F(ab)′2s, scFvs, heavy-light chain dimers, immunoglobulin single variable domains, Nanobodies, domain antibodies, and single chain structures, such as a complete light chain or complete heavy chain. An additional requirement for “activity” of said fragments in the light of the present invention is that said fragments are capable of binding CaSR, preferably with an allosteric effect on CaSR activity in the presence of calcium, and most preferably are (positive) allosteric modulators of CaSR, optimally capable to decrease PTH release in a subject. The term “immunoglobulin (Ig) domain”, or more specifically “immunoglobulin variable domain” (abbreviated as “IVD”) means an immunoglobulin domain essentially consisting of four “framework regions” which are referred to in the art and herein below as “framework region 1” or “FR1”; as “framework region 2” or “FR2”; as “framework region 3” or “FR3”; and as “framework region 4” or “FR4”, respectively; which framework regions are interrupted by three “complementarity determining regions” or “CDRs”, which are referred to in the art and herein below as “complementarity determining region 1” or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1- FR2-CDR2-FR3-CDR3-FR4. It is the immunoglobulin variable domain(s) (IVDs) that confer specificity to an antibody for the antigen by carrying the antigen-binding site. Typically, in conventional immunoglobulins, a heavy chain variable domain (VH) and a light chain variable domain (VL) interact to form an antigen binding site. In this case, the complementarity determining regions (CDRs) of both VH and VL will contribute to the antigen binding site, i.e. a total of 6 CDRs will be involved in antigen binding site formation. In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, with binding to the respective epitope of an antigen by a pair of (associated) immunoglobulin domains such as light and heavy chain variable domains, i.e., by a VH-VL pair of immunoglobulin domains, which jointly bind to an epitope of the respective antigen. An immunoglobulin single variable domain (ISVD) as used herein, refers to a protein with an amino acid sequence comprising 4 Framework regions (FR) and 3 complementary determining regions (CDR) according to the format of FR1-CDR1-FR2-CDR2-FR3-CDR3-FR4. An “immunoglobulin domain” of this invention also refers to “immunoglobulin single variable domains” (abbreviated as “ISVD”), equivalent to the term “single domain antibody” or “single variable domains”, and defines molecules wherein the antigen binding site is present on, and formed by, a single immunoglobulin domain. This sets immunoglobulin single variable domains apart from “conventional” immunoglobulins or their fragments, wherein two immunoglobulin domains, in particular two variable domains, interact to form an antigen binding site. The binding site of an immunoglobulin single variable domain is formed by a single VH/VHH or VL domain. Hence, the antigen binding site of an immunoglobulin single variable domain is formed by no more than three CDR's. As such, the single variable domain may be a light chain variable domain sequence (e.g., a VL-sequence) or a suitable fragment thereof; or a heavy chain variable domain sequence (e.g., a VH-sequence or VHH sequence) or a suitable fragment thereof; as long as it is capable of forming a single antigen binding unit (i.e., a functional antigen binding unit that essentially consists of the single variable domain, such that the single antigen binding domain does not need to interact with another variable domain to form a functional antigen binding unit).

In particular, the immunoglobulin single variable domain may be a Nanobody (as defined herein) or a suitable fragment thereof. Note: Nanobody®, Nanobodies® and Nanoclone® are registered trademarks of Ablynx N.V. (a Sanofi Company). For a general description of Nanobodies, reference is made to the further description below, as well as to the prior art cited herein, such as e.g. described in WO2008/020079. “VHH domains”, also known as VHHs, VHH domains, VHH antibody fragments, and VHH antibodies, have originally been described as the antigen binding immunoglobulin (Ig) (variable) domain of “heavy chain antibodies” (i.e., of “antibodies devoid of light chains”; Hamers-Casterman et al (1993) Nature 363: 446-448). The term “VHH domain” has been chosen to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHHs and Nanobody, reference is made to the review article by Muyldermans (Reviews in Molecular Biotechnology 74: 277-302, 2001), as well as to the following patent applications, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1433793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, by Ablynx N.V. and the further published patent applications by Ablynx N.V. As described in these references, Nanobody (in particular VHH sequences and partially humanized Nanobody) can in particular be characterized by the presence of one or more “Hallmark residues” in one or more of the framework sequences. A further description of the Nanobody, including humanization and/or camelization of Nanobody, as well as other modifications, parts or fragments, derivatives or “Nanobody fusions”, multivalent or multispecific constructs (including some non-limiting examples of linker sequences) and different modifications to increase the half-life of the Nanobody and their preparations can be found e.g. in WO 08/101985 and WO 08/142164. Nanobodies form the smallest antigen binding fragment that completely retains the binding affinity and specificity of a full-length antibody. Nbs possess exceptionally long complementarity-determining region 3 (CDR3) loops and a convex paratope, which allow them to penetrate into hidden cavities of target antigens.

Immunoglobulin single variable domains such as Domain antibodies and Nanobody® (including VHH domains) can be subjected to humanization, i.e. increase the degree of sequence identity with the closest human germline sequence. In particular, humanized immunoglobulin single variable domains, such as Nanobody® (including VHH domains) may be immunoglobulin single variable domains in which at least one amino acid residue is present (and in particular, at least one framework residue) that is and/or that corresponds to a humanizing substitution (as defined further herein). Potentially useful humanizing substitutions can be ascertained by comparing the sequence of the framework regions of a naturally occurring VHH sequence with the corresponding framework sequence of one or more closely related human VH sequences, after which one or more of the potentially useful humanizing substitutions (or combinations thereof) thus determined can be introduced into said VHH sequence (in any manner known per se, as further described herein) and the resulting humanized VHH sequences can be tested for affinity for the target, for stability, for ease and level of expression, and/or for other desired properties. In this way, by means of a limited degree of trial and error, other suitable humanizing substitutions (or suitable combinations thereof) can be determined by the skilled person. Also, based on what is described before, (the framework regions of) an immunoglobulin single variable domain, such as a Nanobody® (including VHH domains) may be partially humanized or fully humanized. Humanized immunoglobulin single variable domains, in particular Nanobody®, may have several advantages, such as a reduced immunogenicity, compared to the corresponding naturally occurring VHH domains. By humanized is meant mutated so that immunogenicity upon administration in human patients is minor or non-existent. The humanizing substitutions should be chosen such that the resulting humanized amino acid sequence and/or VHH still retains the favourable properties of the VHH, such as the antigen-binding capacity. The skilled person will be able to select humanizing substitutions or suitable combinations of humanizing substitutions which optimize or achieve a desired or suitable balance between the favourable properties provided by the humanizing substitutions on the one hand and the favourable properties of naturally occurring VHH domains on the other hand. Such methods are known by the skilled addressee. A human consensus sequence can be used as target sequence for humanization, but also other means are known in the art. One alternative includes a method wherein the skilled person aligns a number of human germline alleles, such as for instance but not limited to the alignment of IGHV3 alleles, to use said alignment for identification of residues suitable for humanization in the target sequence. Also a subset of human germline alleles most homologous to the target sequence may be aligned as starting point to identify suitable humanisation residues. Alternatively, the VHH is analyzed to identify its closest homologue in the human alleles, and used for humanisation construct design. A humanisation technique applied to Camelidae VHHs may also be performed by a method comprising the replacement of specific amino acids, either alone or in combination. Said replacements may be selected based on what is known from literature, are from known humanization efforts, as well as from human consensus sequences compared to the natural VHH sequences, or the human alleles most similar to the VHH sequence of interest. As can be seen from the data on the VHH entropy and VHH variability given in Tables A-5-A-8 of WO 08/020079, some amino acid residues in the framework regions are more conserved between human and Camelidae than others. Generally, although the invention in its broadest sense is not limited thereto, any substitutions, deletions or insertions are preferably made at positions that are less conserved. Also, generally, amino acid substitutions are preferred over amino acid deletions or insertions. For instance, a human-like class of Camelidae single domain antibodies contain the hydrophobic FR2 residues typically found in conventional antibodies of human origin or from other species, but compensating this loss in hydrophilicity by other substitutions at position 103 that substitutes the conserved tryptophan residue present in VH from double-chain antibodies. As such, peptides belonging to these two classes show a high amino acid sequence homology to human VH framework regions and said peptides might be administered to a human directly without expectation of an unwanted immune response therefrom, and without the burden of further humanisation. Indeed, some Camelidae VHH sequences display a high sequence homology to human VH framework regions and therefore said VHH might be administered to patients directly without expectation of an immune response therefrom, and without the additional burden of humanization. Suitable mutations, in particular substitutions, can be introduced during humanization to generate a polypeptide with reduced binding to pre-existing antibodies (reference is made for example to WO 2012/175741 and WO2015/173325), for example at at least one of the positions: 11, 13, 14, 15, 40, 41, 42, 82, 82a, 82b, 83, 84, 85, 87, 88, 89, 103, or 108. The amino acid sequences and/or VHH of the invention may be suitably humanized at any framework residue(s), such as at one or more Hallmark residues (as defined herein) or at one or more other framework residues (i.e. non-Hallmark residues) or any suitable combination thereof. Depending on the host organism used to express the amino acid sequence, VHH or polypeptide of the invention, such deletions and/or substitutions may also be designed in such a way that one or more sites for posttranslational modification (such as one or more glycosylation sites) are removed, as will be within the ability of the person skilled in the art. Alternatively, substitutions or insertions may be designed so as to introduce one or more sites for attachment of functional groups (as described herein), for example to allow site-specific pegylation. In some cases, at least one of the typical Camelidae hallmark residues with hydrophilic characteristics at position 37, 44, 45 and/or 47 is replaced (see WO2008/020079 Table A-03). Another example of humanization includes substitution of residues in FR 1, such as position 1, 5, 11, 14, 16, and/or 28; in FR3, such as positions 73, 74, 75, 76, 78, 79, 82b, 83, 84, 93 and/or 94; and in FR4, such as position 10 103, 104, 108 and/or 111 (see WO2008/020079 Tables A-05-A08; all numbering according to the Kabat).

The term “compound” or “test compound” or “candidate compound” or “drug candidate compound” as used herein describes any molecule, either naturally occurring or synthetic that is designed, identified, screened for, or generated and may be tested in an assay, such as a screening assay or drug discovery assay, or specifically in a method for identifying a compound capable of modulating CaSR activity. As such, these compounds comprise organic and inorganic compounds. For high-throughput purposes, test compound libraries may be used, such as combinatorial or randomized libraries that provide a sufficient range of diversity. Examples include, but are not limited to, natural compound libraries, allosteric compound libraries, peptide libraries, antibody fragment libraries, synthetic compound libraries, fragment-based libraries, phage-display libraries, and the like. Such compounds may also be referred to as binding agents; as referred to herein, these may be “small molecules”, which refers to a low molecular weight (e.g., <900 Da or <500 Da) organic compound. The compounds or binding agents also include chemicals, polynucleotides, lipids or hormone analogs that are characterized by low molecular weights.

Other biopolymeric organic test compounds include small peptides or peptide-like molecules, or derivatives thereof, such as a peptidomimetic containing synthetic amino acids (peptidomimetics) comprising from about 2 to about 40 amino acids, and larger polypeptides comprising from about 40 to about 500 amino acids, such as antibodies, antibody mimetics, antibody fragments or antibody conjugates. The compounds and/or binding agents, including protein binding agents may bind to an allosteric site, which is different from the ligand-binding site, thereby modulating CaSR activity upon ligand/Ca²⁺ binding. Allosteric binding agents increasing the receptor's activity, defines them as positive allosteric modulators (PAMs), including calcimimetics, resulting in increased Calcium (agonist)-sensing activity. Alternatively, compounds acting as negative allosteric modulators (NAMs) of CaSR activity, including calcilytics, result in decreased Calcium(agonist)-sensing activity upon binding an allosteric site. So the term “positive allosteric modulator of Ca²⁺-sensing activity” includes those compounds that are capable to increase CaSR receptor activity in the presence of an agonist such as Ca²⁺.

As used herein, the terms “determining,” “measuring,” “assessing,”, “identifying”, “screening”, and “assaying” are used interchangeably and include both quantitative and qualitative determinations.

The term “subject”, “individual” or “patient”, used interchangeably herein, relates to any organism such as a vertebrate, particularly any mammal, including both a human and another mammal, for whom diagnosis, therapy or prophylaxis is desired, e.g., an animal such as a rodent, a rabbit, a cow, a sheep, a horse, a dog, a cat, a llama, a pig, or a non-human primate (e.g., a monkey). The rodent may be a mouse, rat, hamster, guinea pig, or chinchilla. In one embodiment, the subject is a human, a rat or a non-human primate. Preferably, the subject is a human. In one embodiment, a subject is a subject with or suspected of having a disease or disorder, in particular a disease or disorder as disclosed herein, also designated “patient” herein. However, it will be understood that the aforementioned terms do not imply that symptoms are present. The term “treatment” or “treating” or “treat” can be used interchangeably and are defined by a therapeutic intervention that slows, interrupts, arrests, controls, stops, reduces, or reverts the progression or severity of a sign, symptom, disorder, condition, or disease, but does not necessarily involve a total elimination of all disease-related signs, symptoms, conditions, or disorders.

The term “medicament”, as used herein, refers to a substance/composition used in therapy, i.e., in the prevention or treatment of a disease or disorder. According to the invention, the terms “disease” or “disorder” refer to any pathological state, in particular to the diseases or disorders as defined herein.

This invention also relates to “pharmaceutical compositions” comprising one or more protein binding agents of the invention, and a pharmaceutically acceptable carrier or diluent. These pharmaceutical compositions can be utilized to achieve the desired pharmacological effect by administration to a patient in need thereof. A “pharmaceutically or therapeutically effective amount” of compound or binding agent or composition is preferably that amount which produces a result or exerts an influence on the particular condition being treated. The protein binding agents or the pharmaceutical composition as described herein may also function as a “therapeutically active agent” which is used to refer to any molecule that has or may have a therapeutic effect (i.e. curative or stabilizing effect) in the context of treatment of a disease (as described further herein). Preferably, a therapeutically active agent is a disease-modifying agent, and/or an agent with a curative effect on the disease. By “pharmaceutically acceptable” is meant a material that is not biologically or otherwise undesirable, i.e., the material may be administered to an individual along with the compound without causing any undesirable biological effects or interacting in a deleterious manner with any of the other components of the pharmaceutical composition in which it is contained. A pharmaceutically acceptable carrier is preferably a carrier that is relatively non-toxic and innocuous to a patient at concentrations consistent with effective activity of the active ingredient so that any side effects ascribable to the carrier do not vitiate the beneficial effects of the active ingredient. Suitable carriers or adjuvantia typically comprise one or more of the compounds included in the following non-exhaustive list: large slowly metabolized macromolecules such as proteins, polysaccharides, polylactic acids, polyglycolic acids, polymeric amino acids, amino acid copolymers and inactive virus particles. Such ingredients and procedures include those described in the following references, each of which is incorporated herein by reference: Powell, M. F. et al. (“Compendium of Excipients for Parenteral Formulations” PDA Journal of Pharmaceutical Science & Technology 1998, 52(5), 238-311), Strickley, R. G (“Parenteral Formulations of Small Molecule Therapeutics Marketed in the United States (1999)-Part-1” PDA Journal of Pharmaceutical Science & Technology 1999, 53(6), 324-349), and Nema, S. et al. (“Excipients and Their Use in Injectable Products” PDA Journal of Pharmaceutical Science & Technology 1997, 51 (4), 166-171). The term “excipient”, as used herein, is intended to include all substances which may be present in a pharmaceutical composition and which are not active ingredients, such as salts, binders (e.g., lactose, dextrose, sucrose, trehalose, sorbitol, mannitol), lubricants, thickeners, surface active agents, preservatives, emulsifiers, buffer substances, stabilizing agents, flavouring agents or colorants. A “diluent”, in particular a “pharmaceutically acceptable vehicle”, includes vehicles such as water, saline, physiological salt solutions, glycerol, ethanol, etc. Auxiliary substances such as wetting or emulsifying agents, pH buffering substances, preservatives may be included in such vehicles.

The binding agent or the composition, or pharmaceutical composition of the invention may act as a therapeutically active agent, when beneficial in treating CaSR-related diseases. The binding agent comprising a CaSR-positive allosteric modulator may contain or be coupled to additional functional groups, advantageous when administrated to a subject. Examples of such functional groups and of techniques for introducing them will be clear to the skilled person, and can generally comprise all functional groups and techniques mentioned in the art as well as the functional groups and techniques known per se for the modification of pharmaceutical proteins, and in particular for the modification of antibodies or antibody fragments, for which reference is for example made to Remington's Pharmaceutical Sciences, 16th ed., Mack Publishing Co., Easton, Pa. (1980). Such functional groups may for example be linked directly (for example covalently) to the antibody or ISVD or active antibody fragment, or optionally via a suitable linker or spacer, as will again be clear to the skilled person. One of the most widely used techniques for increasing the half-life and/or reducing immunogenicity of pharmaceutical proteins comprises attachment of a suitable pharmacologically acceptable polymer, such as poly(ethyleneglycol) (PEG) or derivatives thereof (such as methoxypoly(ethyleneglycol) or mPEG). Another technique for increasing the half-life of a binding domain may comprise the engineering into bifunctional or bispecific domains (for example, one ISVD or active antibody fragment against the ECD of CaSR and one against a serum protein such as albumin aiding in prolonging half-life)) or into fusions of antibody fragments, in particular immunoglobulin single variable domains, with peptides (for example, a peptide against a serum protein such as albumin). The coupling to additional moieties will result in a multispecific binding agent, as further disclosed herein.

### DETAILED DESCRIPTION

The present application relates to the identification of positive allosteric modulating nanobodies of the calcium-sensing receptor (CaSR) protein upon specific binding to the extracellularly accessible portion of the receptor, thereby providing for a novel therapeutic route in development of biologicals for targeting CaSR, resulting in reduced PTH levels through specific targeting and elegant modulation of CaSR, which may benefit in eliminating any adverse effects that are currently observed using synthetic drugs, and/or provide for improved PAM compositions potentially used in combination with synthetic drugs.

A first aspect relates to a protein binding agent recognizing the Calcium-sensing receptor (CaSR), and upon binding to its extracellular protein domain, said protein binding agent potently increases the CaSR activity, in an allosteric mode, as compared to the non-bound CaSR receptor. The positive allosteric modulating activity of said protein binding agent may require the presence of calcium, or the presence or binding of another orthosteric ligand which naturally activates the calcium-sensing receptor. The term “allosteric” modulation or activity or regulation as used herein refers to binding at an allosteric or regulatory site, which is a site different from the enzymatically active site or catalytic site of the protein. Positive allosteric modulating activity of a compound refers to the capability of said compound to induce an increase in the activity of the receptor upon binding to an allosteric site, typically in the presence of an agonist or ligand, such as calcium, as compared to the activity of the receptor in the same condition (or bound to the same ligand) but not bound to said compound. Said increase in CaSR activity resulting from the compound binding may refer to an increase of at least 10%, or 20%, or 30%, or even more than 50% as compared to the CaSR receptor not bound to said compound. The allosteric binding agent affects, for instance through the induction of a conformational change of the CaSR protein, the CaSR protein activity. In one embodiment, said protein binding agent acting as a positive allosteric modulator of CaSR activity is a non-naturally occurring protein agent, and is hence different from naturally induced autoantibodies against CaSR in humans. So, in a specific embodiment, said CaSR-specific protein binding agent is a CaSR PAM which is not an autoantibody of a subject, preferably a human, and not an IgG1 antibody. Preferably, said protein binding agent specifically binding CaSR is a heterozygous or exogenous CaSR PAM when present in a cell, an organism, or a subject.

In another embodiment, the protein binding agent specifically binds the CaSR protein when added extracellularly to CaSR-expressing cells. Alternatively, said protein binding agent specifically binds the extracellular calcium binding domain (ECD) of CaSR. On the other hand, it may also occur that the extracellularly accessible portion of the seven-transmembrane (7TM) domain of CaSR is bound by the protein binding agent, or is partially involved in the binding site or epitope. A specific embodiment provides for protein binding agents with positive allosteric modulator activity by binding to a conformational epitope of CaSR, more specifically a conformational epitope present on the extracellular portion of CaSR, which may concern a part or subpart of the ECD, and/or a part or subpart of the 7TM domain. More specifically, said CaSR protein binding agent specific for the extracellular part of CaSR and positively allosterically modulating its activity, in the presence of calcium, may confer binding to a particular epitope only present in calcium-bound CaSR conformation, and confer a novel binding site or epitope. Most preferably, said CaSR protein binding agent is specific for the ECD, and has no binding residues to the 7TM domain.

In fact, it is generally known that G protein-coupled receptors (GPCRs) are a membrane-spanning receptor family that is structurally characterized by the presence of an N-terminal extracellular domain (ECD), an α-helical seven transmembrane domain (7TM) connected via 3 alternating intracellular (ICL1-3) and extracellular loops (ECL1-3) and a C-terminal intracellular domain (ICD) [1]. These structural features allow GPCRs to induce intracellular signaling events in response to extracellular stimuli. The calcium-sensing receptor (CaSR) was discovered as a calcium-regulated channel rather than a GPCR [23].

The cloning of CaSR from isolated bovine parathyroid cells led to the general acceptance of CaSR being a GPCR [24]. CaSR has been shown to primarily couple and signal through G_{q/11} and G_{i/o} proteins upon ligand activation [27]. As implied by its name, the primary ligand of CaSR is ionized calcium (Ca²⁺). In addition to Ca²⁺, however, CaSR can be activated by other multivalent cations (e.g. Mg²⁺, Gd³⁺), cationic polypeptides (e.g. poly-L-lysine), polyamines (e.g. spermine) and aminoglycoside antibiotics (e.g. neomycin) [28]. So an allosteric effect of a protein binding agent may also be obtained in the presence of any of said ligands. Furthermore, the activity of CaSR is also sensitive to allosteric modulation by changes in pH or ionic strength as well as by endogenous or synthetic allosteric ligands [28, 29]. The endogenous allosteric ligands include L-amino acids and γ-glutamyl peptides [28, 30]. The protein binding agents of the present invention, such as antibodies or active antibody fragments, however are not endogenous or naturally-occurring antigen-binding proteins. Synthetic allosteric ligands or binders are known in the art as being developed for therapeutic purposes, though these therapeutics or CaSR PAM drugs do not comprise any antibody-type or immunoglobulin-containing proteins.

The crystal structures of the CaSR ECD homodimer resulted in the identification of an L-amino acid binding site at a location that, in other class C GPCRs, is occupied by the orthosteric ligand, thereby suggesting that L-amino acids and Ca²⁺ may function as co-agonists [40, 42, 43]. This highly conserved binding site is located in the hinge region of the two lobes and was occupied in the CaSR ECD active state crystal structures by L-Trp [46] or an L-Trp derivative [47]. Multiple cation CaSR ECD binding sites were determined, which is in line with the high Hill coefficient of 2-4 obtained with Ca²⁺ in pharmacological assays [51-53]. So, another aspect of the invention relates to a protein complex comprising the CaSR extracellular calcium binding domain (CaSR ECD), and a protein binding agent as disclosed herein, wherein the protein binding agent is bound to the CaSR receptor protein extracellular part. In a particular embodiment, said protein binding agent is an antibody or active antibody fragment, or more particular an ISVD as disclosed herein. In another embodiment, said complex further comprises a ligand, which may be calcium, magnesium, or another naturally-occurring ligand mediating the activity of CaSR. Alternatively, the ligand may be a synthetic or exogenous ligand, competing with the naturally-occurring ligand binding site. In a specific embodiment, said complex is crystalline, and may provide for the 3D-structure obtained for a specific conformation of said CaSR protein complex. Moreover, a purified complex as described herein may be suitable for structural analysis and (conformational) epitope determination of the CaSR-specific allosteric protein binding to the CaSR ECD. Said structural analysis may not require a crystalline complex, as it may involve protein structural analysis via Cryo-electron microscopy (e.g. as in Koehl et al., 2019. Nature, 566:79-84). Other examples to the use of the antibody or active antibody fragments, such as the ISVDs, in complex with the CaSR protein include, but are not limited to, X-ray crystallography, Cryo-EM, identifying or modelling of small molecule drug compounds, screening for small molecules using fragment-based drug discovery, or applying the Nbs in competition experiments, as an alternative to epitope or binding site determination.

In another embodiment, the protein binding agents disclosed herein relate to peptides, peptidomimetics, antibodies, antibody mimetics, single domain antibodies, immunoglobulin single variable domains (ISVDs) or active antibody fragments.

In particular, the protein binding agents disclosed herein relate to antibodies or active antibody fragments specifically binding the CaSR ECD and acting as heterologous PAMs, preferably in the presence of CaSR ligand. A specific embodiment relates to the CaSR ECD-specific antibody or active antibody fragment comprising an ISVD, said ISVD comprising 4 framework regions (FR) and 3 complementarity determining regions (CDR) according to the following formula (1): FR1-CDR1-FR2-CDR2-FR3-CDR3-FR4 (1), and with PAM activity as described herein. More specifically, said antibodies or active antibody fragments comprising ISVDs as disclosed herein comprise a CDR3 region depicted by a sequence selected from SEQ ID NOs:24-30, or a sequence with at least 95% amino acid identity thereof. Indeed, small changes in CDR3 loops have been shown to result in differences in potency, however, limited to maintain the modulation activity of the CaSR receptors. The Nbs or ISVDs belonging to the same family are defined as Nanobodies or ISVDs with a high similarity in their CDR3 sequence (identical length and >80% sequence identity; also see FIG. 10). Nanobodies or ISVDs from the same family derive from the same B-cell lineage and bind to the same epitope on the target. From the examples presented herein, the potency has been shown to be quite similar for Nbs or ISVDs of the same family, and/or showed differences mostly based on differences present within the CDR3.

In a further specific embodiment said ISVDs as disclosed herein contain said CDR3 sequence selected from SEQ ID NO:24-30, and further comprise a CDR1 consisting of a sequence selected from the group of SEQ ID NO: 11-16, and a CDR2 consisting of a sequence selected from the group of SEQ ID NO: 17-23.

A specific embodiment relates to said ISVD comprising a sequence selected from the group of sequences of SEQ ID NO: 3-10, or a sequence with at least 85% amino acid identity thereof. Indeed, as provided by the examples as described below, the same effect, i.e. the same or very similar potency, is obtained for a Nb or ISVD of the same family that has 85% amino acid identity or less over the range of its full length sequence. Another embodiment relates to said ISVD comprising a sequence selected from the group of sequences of SEQ ID NO: 3-10, or a sequence with at least 85% amino acid identity thereof, wherein the 15% variation in sequence identity over the whole length is solely attributed to the residues in the framework regions. Alternatively, said ISVD comprises a sequence selected from a humanized variant of any one of SEQ ID NO: 3-10, which represent the monovalent ISVDs that act as CaSR PAMs as described herein. A further specific embodiment relates to said ISVDs comprising a sequence as depicted in any one of SEQ ID NO:31-34, which represent a non-limiting group of examples of humanized variants of several PAM CaRS ISVDs as described herein. A further embodiment provides for a CaSR PAM antibody or active antibody fragment comprising an ISVD, wherein said ISVD is a further humanized variant of Nb36, Nb4, Nb2, and Nb10, respectively.

Another embodiment of the present invention discloses a multivalent or multi-specific protein binding agent, which comprises said antibody or active antibody fragment as disclosed herein, and a further protein binding agent with the same or a different target specificity, resulting in a multivalent or multispecific agent, respectively. In a preferred embodiment, the antibody or active antibody fragment is operably linked, directly or via a linker, to the further protein binding agent, and/or said further protein binding agent constitutes an antibody or active antibody fragment. Said multispecific binding agent may also be a multivalent binding agent, such as a bivalent binding agent which comprises 2 identical binding agents, or 2 binding agents targeting the same binding site. Said bivalent agents may for instance be two monovalent VHHs targeting the same epitope in the homodimeric CaSR, which may be identical or different VHHs. Multispecific agent may also be biparatopic agents comprising for instance two VHHs targeting different epitopes of the same target, such as the ECD of CaSR. A “multi-specific” form of an ISVD for instance, is formed by bonding together two or more immunoglobulin single variable domains, of which at least one with a different specificity. Non-limiting examples of multi-specific constructs include “bi-specific” constructs, “tri-specific” constructs, “tetra-specific” constructs, and so on. To illustrate this further, any multivalent or multi-specific (as defined herein) protein binding agent of the invention may be suitably directed against two or more different epitopes on the same antigen, for example against epitope 1 and epitope 2 of ECD CaSR; or may be directed against two or more different antigens, for example against CaSR and one as a half-life extension against Serum Albumin. Multivalent or multi-specific binding agents of the invention may also have (or be engineered and/or selected for) increased avidity and/or improved selectivity for the desired CaSR interaction, and/or for any other desired property or combination of desired properties that may be obtained by the use of such multivalent or multi-specific binding agents. For instance, the combination of one or more ISVDs binding epitope 1, and one or more ISVDs binding epitope 2 as described herein, results in a multi-specific binding agent of the invention. Said multi-specific binding agent comprises at least said binding agents directed against epitope 1 and epitope 2, which may be coupled via a linker, spacer. Typically these are generated as recombinantly produced fusion proteins conjugated via a glycine-serine or PEG-like amino acid linker. Upon binding CaSR, said multi-specific binding agent or multivalent ISVD may have an additive or synergistic impact on the CaSR activity. The protein binding agents of the invention may be coupled to a functional moiety, a targeting moiety, a half-life extending moiety, or to a cell penetrant carrier.

Therapeutic Potential

Parathyroid hormone (PTH) is synthesized and secreted from the parathyroid gland chief cells and directly affects the serum Ca²⁺ concentration by acting on the kidney, bone and indirectly on the intestine. PTH triggers Ca²⁺ release from the bone, induces Ca²⁺ reabsorption from the kidneys and stimulates the conversion from inactive 25-hydroxyvitamin D (25(OH)D) into the active form 1.25-dihydroxyvitamin D (1,25(OH)2D) in the kidneys. The active 1,25(OH)2D further promotes Ca²⁺ release from the bone and stimulates Ca²⁺ absorption by the intestine. All together, these PTH-mediated effects lead to an increase in the serum Ca²⁺ concentration. Calcitonin is synthesized and secreted from the thyroid gland C-cells. PTH and calcitonin have opposing effects and thus calcitonin leads to a decrease in the serum Ca²⁺ concentration. The activity of CaSR controls the synthesis and secretion of PTH and calcitonin. CaSR activity increases when serum Ca²⁺ concentration increases or exceeds the physiological range. The activated CaSR suppresses the synthesis and secretion of PTH, while simultaneously stimulating the synthesis and secretion of calcitonin. The opposite occurs when the Ca²⁺ concentration falls below the physiological range and CaSR adopts the inactive state [54, 55]. Consequently, many naturally occurring CaSR mutations have been linked to disorders underlying a dysregulation in this process [56, 57].

In a particular embodiment, the protein binding agent or pharmaceutical composition as described herein, is capable of reducing parathyroid hormone (PTH) levels in vivo, as for instance in healthy animals, such as rats, as well as in animal disease models. Specific embodiments relate to the protein binding agent or the pharmaceutical compositions as described herein, for use as in treatment of a subject to reduce parathyroid hormone secretion. Preferably said subject being human.

Its prominent role in calcium homeostasis makes CaSR a well-established drug target in calcium homeostasis disorders. In the 1990's, the phenylalkylamine compounds NPS R-568 and its derivative NPS R-467 were developed as the first synthetic allosteric ligands or compounds acting on CaSR [58, 59]. By increasing the sensitivity of CaSR for extracellular Ca²⁺, these positive allosteric modulator (PAM) small molecules potentiated the Ca²⁺-mediated activity of CaSR on the inhibition of PTH secretion and the potentiation of calcitonin secretion [59]. Unfortunately, clinical development of NPS R-568 ceased due to the observed low oral bioavailability (<5%) and high first-pass metabolism by CYP2D6 which could not be accomplished in 5-7% of the patient population [58, 60]. Optimization of the NPS R-568 and NPS R-467 structure led to the discovery of cinacalcet. Cinacalcet presents an oral treatment for disorders underlying hyperparathyroidism [64-66], but its clinical utility is unfortunately restricted due to the high risk of gastrointestinal adverse effects (i.e. nausea and vomiting) and hypocalcemia [58, 67]. Other phenylalkylamine PAMs have been developed for CaSR, evocalcet, and the octapeptide etelcalcetide, both with fewer gastrointestinal side effects than cincalcet [71] [72] [73], though hypocalcemia could not be avoided and thus treatment with currently available CaSR PAM small molecules remains sub-optimal [74, 75].

A further embodiment provides for a pharmaceutical composition comprising the antibody or active antibody fragment, or the multispecific binding agent as disclosed herein.

Alternatively, a pharmaceutical composition is disclosed comprising the CaSR-specific allosteric antibody or active antibody fragment as presented herein, further comprising a synthetic compound known to have PAM CaSR activity. Specifically, said further synthetic compound may be one or more compounds selected from the group of NPS R-568, NPS R-467, cinacalcet, evocalcet, and etelcalcetide, or analogues or derivatives thereof. Another specific embodiment relates to said pharmaceutical composition wherein the antibody or active antibody fragment as disclosed herein comprises an ISVD with a CDR1, 2 and 3 sequence selected tom the CDRs of Nb2, Nb4, or Nb10, and comprising a synthetic CaSR PAM. More specifically said pharmaceutical composition comprises at least one antibody or active antibody fragment comprising an ISVD selected from the group of SEQ ID NO: 3, 5, 9, 32, 33, or 34, or from a humanized variant of any one thereof, and further comprises a small molecule CaSR PAM. These pharmaceutical compositions comprising a combination of said the at least one antibody or active antibody fragment as disclosed herein, and a chemical or peptide known to act as a PAM on CaSR in the presence of Calcium may provide for an improved drug composition. Indeed, the combination of such 2 types of PAMs of CaSR seem to act in a synergistic agonism, also in the absence of calcium ligand, potentially due to presence of alternative CaSR ligand, and acting dependent on the Ca²⁺-mediated CaSR activity (as shown by the S170 CaSR mutant; see Examples). It may also be envisaged herein that a PAM binding exclusively to the ECD, preferably an immunoglobulin-based agent, in combination with a PAM binding to the 7TM CaSR domain, preferably a small molecule, provides for a synergistic allosteric modulatory effect which surprisingly also activates CaSR in the absence of extracellularly added calcium ligand, thereby opening new avenues in therapeutic treatments.

Another aspect of the invention relates to the antibody or active antibody fragment protein binding agents, multispecific binding agents, or the pharmaceutical compositions as disclosed herein, for use as a medicament. Besides the calcium-related function, the expression of CaSR in tissues and cells unrelated to calcium homeostasis (e.g. lungs, heart, intestine, pancreatic islets, tumour cells, spermatozoa) proposes the usage of CaSR PAMs in various other pathologies. As a non-limiting example, treatment with CaSR PAMs has been proposed to be beneficial in neuroblastoma as CaSR was found to be epigenetically silenced in malignant neuroblastomic tumour cells [83, 84].

Another embodiment so relates to the antibody or active antibody fragment protein binding agents, multispecific binding agents, or the pharmaceutical compositions as described herein, for use in treatment of CaSR-related disorders. Said disorders are known to a skilled person, and commonly provided for in the public knowledge, and include, but are not limited to, hypercalcemia disorders, such as secondary and primary hyperparathyroidism, digestive diseases, respiratory diseases, cardiovascular and neoplastic disorders.

It is to be understood that although particular embodiments, specific configurations as well as materials and/or molecules, have been discussed herein for methods, samples and biomarker products according to the disclosure, various changes or modifications in form and detail may be made without departing from the scope of this invention. The following examples are provided to better illustrate particular embodiments, and they should not be considered limiting the application. The application is limited only by the claims.

### EXAMPLES

### Example 1. Generation of CaSR Nanobodies Using Whole Recombinant Cells

Nanobodies against the human calcium-sensing receptor (hCaSR) were generated using whole recombinant cells to ensure native receptor conformation. A llama was immunized with whole Flp-In CHO cells recombinantly expressing myc-tagged wild-type hCaSR (Flp-In CHO myc-hCaSR WT). A phage display library was constructed from the isolated nanobody genes using the pMESy4 phagemid. Subsequent nanobody selections were performed. Nanobody phage display selections were performed in the presence of 10 mM CaCl₂ or in the presence of 5 μM negative allosteric modulator (NAM) NPS 2143 supplemented with 0.5 mM EDTA in an attempt to select nanobodies that recognize the active and inactive state of CaSR, respectively. All materials (i.e. Eppendorf tubes, FACS tubes, cells and input phages) used were pre-blocked in 1% BSA and 10% FCS for at least 30 minutes to avoid non-specific surface binding of the phage. Moreover, the selection was performed at 4° C. to prevent receptor internalization. The phages were eluted from the cells, rescued in TG1 bacterial cells after which their nanobody DNA was extracted and sequenced by Sanger sequencing. This selection procedure was performed in two consecutive rounds. Nanobody DNA sequences obtained after the first and second round were analysed and grouped in families according to their sequence identity. As expected, certain sequences were found more frequently after the second round of selection indicating an enrichment of these binders. A nanobody sequence library was composed with nanobody sequences across different families. A total of 37 nanobody sequences from 25 families were selected for purification and pharmacological characterization. Within this library, 10 nanobodies were obtained with the CaCl₂ selection, 14 nanobodies were obtained with the NPS 2143/EDTA selection and 13 nanobodies were found in both selections.

In conclusion, we successfully created a llama-derived CaSR nanobody library for the first time. Pharmacological characterization led to the discovery of 8 nanobodies acting as potent CaSR PAMs, of which 5 belong to different sequence families. In agreement with their pharmacological profile, the 8 PAM-acting nanobodies were all solely obtained from the 10 mM CaCl₂ selection. No clear NAM activity was obtained from the nanobodies derived from the 5 μM NPS 2143 supplemented with 0.5 mM EDTA panning condition.

### Example 2. Pharmacological Characterization of CaSR Nanobodies

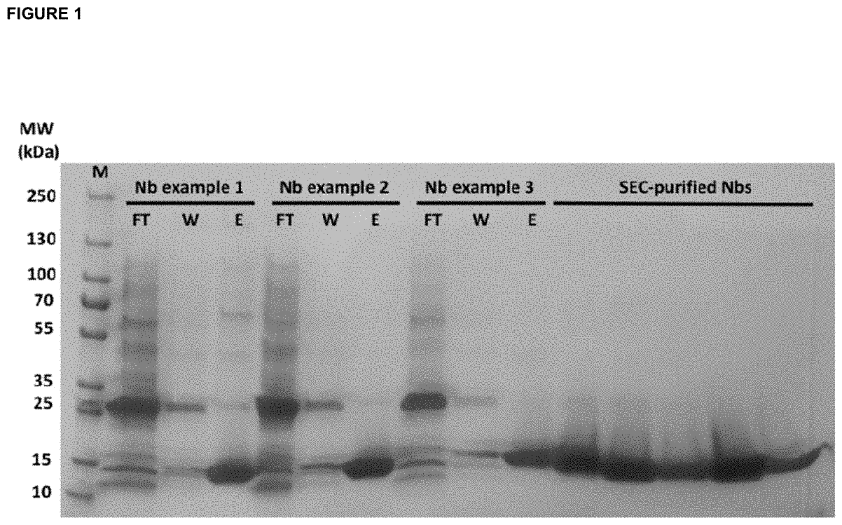

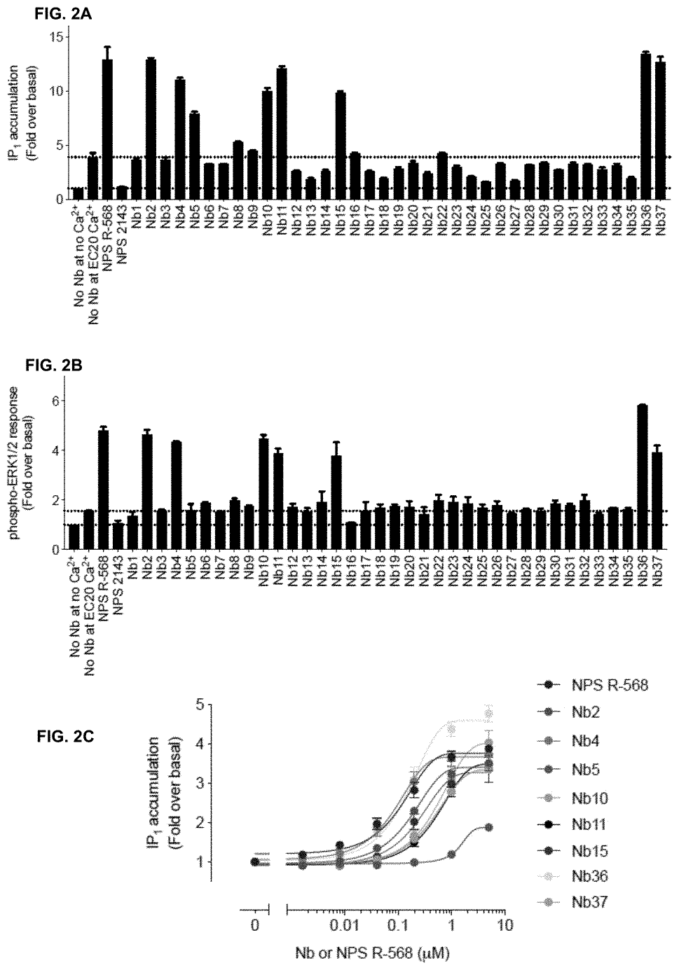

Nanobodies were successfully purified by immobilized metal ion affinity chromatography (IMAC) and size exclusion chromatography (SEC) prior to pharmacological characterization (FIG. 1). Each nanobody was initially screened for G_{q/11} activity in the Cisbio IP₁ accumulation assay as this is the primary signaling pathway of CaSR (Chavez-Abiega et al., 2020). Whole HEK293 HA-hCaSR WT cells were pre-incubated for 30 minutes at 37° C. with a fixed nanobody concentration (5 μM) prior to stimulation with the endogenous ligand Ca²⁺. The well characterized positive allosteric modulator (PAM) chemical NPS R-568 and negative allosteric modulator (NAM) chemical NPS 2143 were added as reference compounds. As shown in FIG. 2A, 8 nanobodies (i.e. CaSR Nb2, Nb4, Nb5, Nb10, Nb11, Nb15, Nb36 and Nb37) potentiated IP₁ accumulation in the presence of EC₂₀ Ca²⁺. No nanobodies with agonist or NAM activity were observed when all nanobodies screened in the absence or in the presence of EC₈₀ Ca²⁺, respectively (FIGS. 4A and 4B). We next screened the library for ERK1/2 phosphorylation, a downstream signalling event that was shown to be predominantly G_{q/11}-dependent with the selective G_{q/11} YM-254890 inhibitor (FIG. 5A). Except for CaSR Nb5, the same nanobodies acted as PAMs when ERK1/2 phosphorylation was measured in the presence of EC₂₀ Ca²⁺ (FIG. 2B). Again, none of the nanobodies elicited an agonist or NAM response (FIGS. 5B and 5C). The 8 pharmacologically active nanobodies potentiated IP₁ accumulation at EC₂₀ Ca²⁺ in a concentration dependent manner with a potency rank order of CaSR Nb4>NPS R-568>CaSR Nb36>CaSR Nb2>CaSR Nb15>CaSR Nb10>CaSR Nb11>CaSR Nb37>>>CaSR Nb5 (FIG. 2C).

These pharmacologically active nanobodies were all obtained with the CaCl₂ selection and could be divided into 5 sequentially distinct nanobody families. CaSR Nb4 was highly similar to CaSR Nb11 (89.9% sequence identity) and CaSR Nb36 (90.6% sequence identity), and CaSR Nb10 was highly similar to CaSR Nb37 (97.8% sequence identity). Therefore, further pharmacological characterization was performed with the most potent nanobody of each family (i.e. CaSR Nb2, Nb4, Nb5, Nb10 and Nb15). The nanobody and NPS R-568 PAM activity did not require pre-incubation, thus further characterization was executed without the 30 minutes pre-incubation step (FIG. 6).

We were able to confirm the PAM activity of the most potent nanobodies in the orthogonal phospho-ERK1/2 assay, which we showed also detect G_{q/11} signaling activity. Only the PAM activity of CaSR Nb5 could not be confirmed which however aligns with it being the weakest PAM in the IP₁ assay (FIG. 2C). Where the phospho-ERK1/2 assay measures a natural cell response (i.e. phosphorylation of ERK1/2), the IP1 accumulation assay measures a non-natural accumulation of the second messenger IP₁. This difference in read-out makes the IP1 accumulation assay more sensitive to picking up activity from low potent ligands such as Nb5.

To further characterize the PAM activity of the nanobodies, we performed Ca²⁺ concentration-response curves in the presence of varying concentrations of cinacalcet or nanobody Nb4 (FIG. 12). Increasing concentrations of either cinacalcet or nanobody Nb4 potentiate the Ca²⁺ response and lead to a leftward shift of the concentration-response curves. These data demonstrate that nanobody Nb4 is a positive allosteric modular with a pharmacological profile similar to the small molecule PAM cinacalcet.

### Example 3. Several CaSR Nanobody and NPS R-568 Combinations Elicit a Synergistic Agonist Effect

Potential synergy between two CaSR nanobodies or one CaSR nanobody and NPS R-568 was investigated in the IP1 accumulation assay (FIG. 3A and FIG. 3B). The inactive nanobody CaSR Nb7 was combined with the nanobodies or NPS R-568 to ensure equal ligand exposure (i.e. 10 μM) in every tested condition. No clear synergy was observed between the CaSR nanobody/nanobody and CaSR nanobody/NPS R-568 combinations at EC₂₀ Ca²⁺ (FIG. 3A). Interestingly, when no Ca²⁺ was added, an increase in IP₁ accumulation was observed for NPS R-568 combined with CaSR Nb2, Nb4 or Nb10 (FIG. 3B). Besides Ca²⁺, CaSR can also be activated by other physiological ligands including other multivalent cations and proteinogenic L-amino acids (Brown et al., 1990; Conigrave et al., 2000; Handlogten et al., 2000). These physiological ligands are difficult to completely remove from an experimental setup for two reasons: 1) they are vital components in assay buffers and cell culture media and 2) they can be released from the cell either through passive or active membrane transport (Meier et al., 2002; Brini and Carafoli, 2011). To investigate whether these CaSR nanobody/NPS R-568 combinations show a synergistic agonist or PAM effect, two approaches were used. First, the CaSR nanobody/NPS R-568 combinations were tested in the presence of 0.5 mM EDTA to chelate ambient extracellular cations (FIG. 3C). We previously demonstrated that this EDTA concentration had no non-specific effect on HEK293T cells (Mos et al., 2019). As presented in FIG. 3C, 0.5 mM EDTA effectively decreased the EC₂₀ Ca²⁺-induced response, while the buffer (i.e. no added Ca²⁺) response remained unaffected. The presence of 0.5 mM EDTA did not eliminate the observed increase in IP₁ accumulation for the different CaSR nanobody/NPS R-568 combinations. These results indicate that the increase in IP₁ accumulation observed for these CaSR nanobody/NPS R-568 combinations is not caused by ambient extracellular cations. Second, CaSR nanobody/NPS R-568 combinations were applied to HEK293A cells transiently transfected with the CaSR loss-of-function mutant S170A. The S170 residue is located within the aromatic L-amino acid binding site in the extracellular domain (ECD) of CaSR. Crystal structures from the CaSR ECD revealed that this residue directly interacts with L-Trp (Geng et al., 2016; Zhang et al., 2016). Mutating this residue to an alanine has previously been shown to severely impair Ca²⁺-induced IP₁ accumulation of CaSR (Bräuner-Osborne et al., 1999; Geng et al., 2016). As shown in FIG. 3D, EC₂₀ Ca²⁺-mediated IP₁ accumulation is abolished in cells transiently transfected with HA-hCaSR S170A. Moreover, none of the CaSR nanobody/NPS R-568 combinations induced IP₁ accumulation in hCaSR S170A.

In conclusion, a synergistic agonist response was observed when NPS R-568 was co-stimulated with CaSR Nb2, Nb4 or Nb10. When applied alone, the pharmacologically active CaSR nanobodies and reference PAM NPS R-568 did not induce IP₁ accumulation in the absence of added Ca²⁺. When combined, an increase in IP₁ accumulation was observed for NPS R-568/Nb2, NPS R-568/Nb4 and NPS R-568/Nb10. The chelating agent EDTA did not abolish the observed increase in IP₁ accumulation, indicating a synergistic agonist effect between NPS R-568 and these three nanobodies. Interestingly, none of the CaSR nanobody/NPS R-568 combinations induced IP₁ accumulation in cells expressing the CaSR S170A loss-of-function mutant.

All together, we propose that (1) L-amino acid binding rather than Ca²⁺ binding is necessary to obtain the NPS R-568/nanobody agonist responses or (2) the S170A mutation shifts the conformational state of CaSR towards the inactive conformation.

### Example 4. Screening Verification and Rat CaSR Ortholog Selectivity Testing

The PAM activity of the 8 potent CaSR nanobodies identified in the initial screen was confirmed in subsequent experiments. These CaSR nanobodies potentiated IP₁ accumulation of EC₂₀ Ca²⁺ in a concentration-dependent manner (FIG. 7A). The potencies and IP₁ maximum response corresponding to the concentration-response curve in FIG. 7A are presented in Table 1. The potencies of CaSR Nb4 and CaSR Nb36 were comparable to the potency of the small molecule PAM NPS R-568.

The 8 PAM-acting CaSR nanobodies originate from 5 nanobody families. CaSR Nb10 and CaSR Nb37 belong to the same family, which is also the case for CaSR Nb4, CaSR Nb11 and CaSR Nb36. The concentration-response curves from the nanobodies within these shared families are shown separately in FIGS. 7B and 7C. There are no apparent differences between the CaSR Nb10 and CaSR Nb37 concentration-response curves (FIG. 7B). These nanobodies differ at 3 positions within their protein sequence of which none are within the CDR3 region. Interestingly, differences in potency and maximum response are observed for CaSR Nb4, CaSR Nb11 and CaSR Nb36 (FIG. 7C). Based on sequence analysis, the lower potency of CaSR Nb11 could be caused by 2 amino acids within CDR3 and/or 1 amino acid positioned before the start of CDR3, while 3 other amino acids within CDR3 could be responsible for the greater maximum response of Nb36. These results demonstrate that minor changes in the CDR3 region of the Nb sequence can affect pharmacological activity. Mutagenesis studies could be applied in the future to improve potency and/or efficacy.

The rat and human CaSR orthologs have an overall sequence identity of 93.6% and an ECD sequence identity of 95.7% (SEQ ID NO:1 aligned with SEQ ID NO:2). Thus, differences in CaSR nanobody activity are unlikely to be observed. Indeed, all 8 nanobodies potentiated IP₁ accumulation at EC₂₀ Ca²⁺ with potencies comparable to the potencies obtained with the human ortholog (FIG. 7D and Table 1). Besides CaSR Nb36, similar results were obtained among the nanobodies that originate from the same family (FIGS. 7E and 7F). For Nb36, the increase in maximum response relative to Nb4 and Nb11 previously observed with human CaSR was absent (FIGS. 7C and 7F). The binding site of these nanobodies still remains to be found. The difference in CaSR Nb36 maximum response and the high interspecies sequence identity could bring us one step closer to finding the binding site for CaSR Nb4, Nb11 and Nb36. Overall, these results indicate that these nanobodies can be further characterized in in vivo rat models.

TABLE 1
CaSR Nanobody potency determination for human and rat CaSR.

| | Human ortholog | | Rat ortholog | |
|---|---|---|---|---|
| | | Max. response | | Max. response |
| | Potency | (fold over | Potency | (fold over |
| | (pEC₅₀ ± S.E.M.) | basal ± S.E.M.) | (pEC₅₀ ± S.E.M.) | basal ± S.E.M.) |
| NPS R-56S | 6.92 ± 0.15 | 3.89 ± 0.07 | 6.59 ± 0.22 | 3.43 ± 0.76 |
| Nb2 | 6.71 ± 0.08 | 3.52 ± 0.21 | 6.46 ± 0.07 | 3.01 ± 0.51 |
| Nb4 | 7.08 ± 0.13 | 3.74 ± 0.08 | 7.09 ± 0.09 | 3.03 ± 0.63 |
| Nb5 | N.D.* | 1.88 ± 0.05 | N.D.* | 1.28 ± 0.12 |
| Nb10 | 6.33 ± 0.03 | 3.36 ± 0.09 | 6.29 ± 0.02 | 2.89 ± 0.13 |
| Nb11 | 6.28 ± 0.07 | 3.50 ± 0.17 | 6.24 ± 0.11 | 2.90 ± 0.23 |
| Nb15 | 6.59 ± 0.03 | 3.38 ± 0.36 | 6.31 ± 0.07 | 2.88 ± 0.13 |
| Nb36 | 6.78 ± 0.17 | 4.78 ± 0.21 | 6.78 ± 0.03 | 3.10 ± 0.12 |
| Nb37 | 6.22 ± 0.05 | 4.04 ± 0.32 | 6.00 ± 0.28 | 3.07 ± 0.39 |

Potency values as pEC50 and maximum response (max. response) as fold over basal of the nanobodies and small molecule PAM NPS R-568 obtained for human CaSR (corresponding to FIGS. 7A-C) and rat CaSR (corresponding to FIGS. 7D-F). Nb numbers correspond to CaSR Nbs as mentioned herein (SEQ ID Nos: 3-10); ND, Not determined.
*Greater than the highest tested concentration.

### Example 5. CaSR Nb4 is Capable of Increasing the Ca²⁺ Potency in Pathological CaSR Conditions

In a preliminary experiment, we investigated the impact of the most potent nanobody on the pathological CaSR mutant Q459R. In comparison with loss-of-function mutant S170A, Q459R is a naturally occurring loss-of-function mutant that has previously been associated with familial hypocalciuric hypercalcemia (FHH). The effect of increasing concentrations of CaSR Nb4 or the clinically used PAM cinacalcet on the Ca²⁺ concentration-response curve were measured with the IP1 accumulation assay. As observed in FIG. 8, both cinacalcet and CaSR Nb4 are able to increase the potency for Ca²⁺. The PAM effect of CaSR Nb4 (0.04 μM) is initiated at a much lower concentration than for cinacalcet (5 μM).

### Example 6. Delineate the Nanobody CaSR Binding-Site and Molecular Mode-of-Action

For the seven different nanobodies with potencies in the 10 to 600 nM range, further characterization of their recognition site on the CaSR antigen was performed, since the identification of the nanobody binding-sites at the CaSR is key to understand their interaction and mode-of-action at a molecular and structural level, and will further aid in the development of second-generation nanobodies with enhanced pharmacological parameters. Nanobodies belonging to each family of the five different nanobody families are expected to interact with distinct regions of the CaSR. To determine if the nanobodies bind to the canonical allosteric binding site in the 7TM domain, we synthesized the tritiated small molecule [³H]evocalcet PAM and found that the nanobodies (exemplified by Nb4 in FIG. 11A) do not displace the radioligand from its binding site in the 7-transmembrane domain, while other small molecules known to bind via the 7TM domain, such as cinacalcet and NPS-2143 did displace the labelled evocalcet (FIG. 11A), allowing to conclude that the Nbs do not bind the same site as evocalcet, and most likely thus not bind the 7TM domain, and thus recognize a different binding site as compared to the conventionally known small molecule calcimimetics and calcilytics.

In addition, we used surface plasmon resonance to show that Nb4 binds to the isolated purified extracellular ‘Venus flytrap domain’ of CaSR and that the binding affinity is dependent on the presence of Ca²⁺ and/or L-Trp, and synergistically increases upon the presence of both ligands (FIG. 11B).

The same analysis is performed for the remaining nanobodies in the surface plasmon resonance assay. Furthermore, chimera between the CaSR and the homologue receptor mGluR5 generation allows to identify nanobody epitope sites [85-87]. CaSR chimeras with mGluR5 in which the extracellular amino terminal domain, 7-transmembrane domain or individual extracellular loops of CaSR have been replaced with the corresponding domains of mGluR5 allow to pinpoint exact nanobody binding modes. Finally, nanobodies in complex with CaSR are used for cryo-EM studies to build on the previously revealed preliminary cryo-EM structures of the full-length CaSR, to finally validate the binding sites by for instance mutational analysis in combination with SPR and the pharmacological assays described herein.

### Example 7. Humanized Variants Tested in Potency and for Further Substitutions

In order to develop the most promising Nb candidates further into biologicals for use in treatment of human subjects, the Nb sequences are known to require humanization substitutions, as described previously herein, and as known by the skilled person.

As a non-limiting example of a humanization, a number of humanized variants is provided herein for the most potent Nbs in SEQ ID NO:31-34, representing humanized variant 1 of Nb36, Nb4, Nb2, and Nb10, respectively. The variants comprise CDR regions which are identical to the originally identified llama VHHs, as to retain the binding affinity and related PAM functionality to CaSR, but have been modified in line with human germline sequences, in line with and based on expertise showing which substitutions are critical for good pharmacological profiling and improved biophysical properties of the Nbs. Despite the knowledge in the art on how to perform humanization substitution, the potency of these humanized variants needs to be analyzed, and in case that an undesired effect is observed, reversion of one or more amino acid substitutions to its original residue may be envisaged to obtain the most preferred humanization variant.

Furthermore, although CDRs have been retained in their sequence as originally identified for the llama Nbs, the CDR2 residues of Nb4_h1 may require further substitutions as to avoid Asn deamidation (position 54-55 NG of SEQ ID NO:32) (Nb4h1), as well as the Methionine in CDR2 of Nb10_h1 (position 51 of SEQ ID NO: 34), may require further substitution as to avoid Met oxidation. Though, since such CDR substitutions may impact the target binding, and further humanization would thus be conditional on retained binding affinity, these should be determined experimentally as to develop the most preferred humanization variant of Nb4 and Nb10.

### CONCLUSIONS

The present invention provides for a panel of Nanobodies with unknown potential to aide in several applications. Firstly, Nanobodies have been shown to perform as excellent stabilizing chaperones in structural analysis. Indeed, they unveil novel potential drug targeting sites and act as chaperones in GPCR crystallization (Heukers et al., 2019). The first full-length structure of a class C metabotropic glutamate receptor subtype 5 homodimer has recently been resolved with the help from a pharmacologically active nanobody which potentiated agonist binding (Koehl et al., 2019). For CaSR only the extracellular domain structure has been resolved (Geng et al., 2016; Zhang et al., 2016). The nanobodies described in this study offer the opportunity to facilitate the determination of an active conformation full-length structure of CaSR. Secondly, depending on the mechanism of adverse effects of the current CaSR drugs, the CaSR nanobodies could have a potential for lowered adverse effects. The main drawback of current CaSR-targeting PAMs is the high risk for severe hypocalcemia and gastro-intestinal adverse effects (Bover et al., 2016; Fukagawa et al., 2018; Patel and Bridgeman, 2018). CaSR is a widely expressed GPCR and thus adverse effects through activation of CaSR in different tissues such as the gastro-intestinal tract is not surprising (Chavez-Abiega et al., 2019). Nanobodies could potentially overcome these adverse effects by conjugation to other moieties, e.g. antibodies/nanobodies binding tissue-specific proteins, to allow regional selectivity. Thirdly, the observed synergy between the nanobodies with NPS R-568, a PAM structurally related to cinacalcet and evocalcet, proposes a novel therapeutic strategy where co-application or combination therapy of a nanobody with a low dose of synthetic PAM could potentially reduce these adverse effects, and/or improve treatment options.

Materials and Methods

Materials

Unless stated otherwise, general reagents were purchased from Sigma-Aldrich (St. Louis, Mo., USA) and cell culture reagents were bought from Thermo Fisher Scientific (Waltham, Mass., USA). The positive allosteric modulator NPS R-568 hydrochloride was purchased from Tocris Bioscience (Bristol, UK), while the negative allosteric modulator NPS 2143 hydrochloride was synthesized in-house at the University of Copenhagen, Denmark as previously described (Johansson et al., 2013). The G_{q/11} inhibitor YM-2548090 was bought from Wako Chemicals GmbH (Neuss, DE). Nanobodies were extracted from bacterial cells with TES buffer: 50 mM Tris pH 8.0, 1 mM EDTA, 150 mM NaCl, 20% sucrose (Merck, Darmstadt, DE), 0.4 mg/mL lysozyme, 0.1 mg/mL AEBSF (Carl Roth, Karlsruhe, DE) and 1 μg/mL Leupeptin hemisulphate (Carl Roth, Karlsruhe, DE). Nanobody selections and pharmacological characterization were performed in assay buffer: Hank's Balanced Salt Solution without Ca²⁺, Mg²⁺ and phenol red (HBSS, Thermo Fisher Scientific, Waltham, Mass., USA) supplemented with 20 mM HEPES and adjusted to pH 7.4.

CaSR Plasmid DNA Constructs

The human CaSR wild-type (hCaSR WT) sequence (common Q1011 variant of uniport identifier P41180; SEQ ID NO: 1) was cloned in a pcDNA5 FRT vector to allow stable cell line generation according to the Flp-In technology. The final pcDNA5 FRT hCaSR constructs contained an upstream mGlu5 cleavable signal peptide followed by an N-terminal HA or myc tag. The S170A mutation was introduced using the Quickchange II site-directed mutagenesis kit (Agilent technologies, Santa Clara, Calif., USA). The rat CaSR (rCaSR) WT (uniprot identifier: P48442; SEQ ID NO:2) construct was made as previously described (Jacobsen et al., 2017). This construct consists of a mGlu5 signal peptide and HA tag upstream of the rCaSR WT sequence in a pEGFPN1 background vector.

Stable Cell Line Generation

The Flp-In technology was applied to establish Flp-In human embryonic kidney 293 (Flp-In HEK293, RRID: CVCL_U421) and Flp-In Chinese hamster ovary (Flp-In CHO, RRID: CVCL_U424) cell lines stably expressing hCaSR WT. At ˜70% confluence, Flp-In HEK293 and Flp-In CHO cells were transfected with a total of 8 μg/dish at a pcDNA5 FRT hCaSR:pOG44 plasmid DNA ratio of 1:9 and 20 μL/dish Lipofectamine 2000. Cells transfected without plasmid DNA were included as negative control. 24 h after transfection, the cells were split 1/10, 1/20 and 1/30 into fresh p10 dishes. As from 48 h after transfection, the cells were kept under continuous selection pressure with 200 μg/mL hygromycin B for the Flp-In HEK293 cells and 500 μg/mL hygromycin B for the Flp-In CHO cells. Colonies resistant to hygromycin B were pooled together to obtain polyclonal stable Flp-In HEK293 and Flp-In CHO cell lines. Cell line validation data of the polyclonal Flp-In CHO myc-hCaSR WT and Flp-In HEK293 HA-hCaSR WT cell lines used in this study are presented in FIGS. 9A-9G. A Flp-In HEK293 cell line stably expressing the myc-GABAA δ subunit previously prepared in a similar fashion was used in this study as non-CaSR expressing control cell line (Falk-Petersen et al., 2017). The GABAA δ subunit is not functional and not present on the cell-surface when expressed alone.

Llama Immunization with hCaSR WT Recombinantly Expressed in Flp-In HEK293 Cells

CaSR-specific nanobodies were generated as previously described (Pardon et al., 2014) with some adjustments. In brief, Flp-In CHO myc-hCaSR WT cells were harvested and stored in aliquots of 1E7 cells/aliquot in liquid N2. One llama (Lama glama) was immunized for 6 weeks. Weekly, Flp-In CHO myc-hCaSR WT cells (8E7-3E7 cells/injection) were thawed, washed twice with ice cold HBSS buffer and injected subcutaneously. Four days after the final boost, blood was taken to isolate peripheral blood lymphocytes. From these lymphocytes, RNA was purified and reverse transcribed by PCR to obtain cDNA. The resulting library was cloned into the phage display vector pMESy4 bearing a C-terminal hexa-His tag and a CaptureSelect sequence tag (Glu-Pro-Glu-Ala). A Nanobody phage display library of 2E9 independent clones was obtained. The phage library was prepared as previously described (Pardon et al., 2014).

Nanobody Purification

Nanobodies selected for pharmacological characterization were soluble expressed in the periplasm of E. coli WK6 cells, extracted and purified by immobilized metal ion affinity chromatography (IMAC) followed by size exclusion chromatography (SEC). First, 5 μL pMESy4 construct containing the desired nanobody sequence was transformed into E. coli WK6 competent cells. The pMESy4 construct and bacterial cells were mixed gently and placed on ice for 20 minutes after which the cells were subjected to a heat shock for 1.5 minutes at 42° C. The heat shocked cells were cooled on ice for 2 minutes and transferred to an Eppendorf tube containing 900 μL LB media. The mixture was shaken at 180 rpm for 1 h at 37° C., plated on a LB agar plate containing 100 μg/ml ampicillin and incubated overnight at 37° C. Second, a pre-culture was prepared in the afternoon by inoculating a single colony in 5 mL LB media containing 100 μg/ml ampicillin. The pre-culture was grown overnight at 37° C. and 180 rpm. The next morning, 3.5 mL pre-culture was added to 1 L TB media containing 100 μg/ml ampicillin and incubated at 37° C. and 120 rpm for at least 7 hours. The remainder of the pre-culture was mini-prepped and sent for sequencing to confirm the nanobody DNA sequence. At the end of the day, 1 mM isopropyl 3-D-1-thiogalactopyranoside (IPTG) was added to the flask to induce nanobody expression overnight at 28° C. and 120 rpm. The induced E. coli WK6 competent cells were centrifuged for 10 minutes at 5,000 rpm and the pellet was dissolved in TES buffer to start the lysozyme extraction. The dissolved pellet was supplemented with 5 mM MgCl2 and 1:1000 DNAse I (2500 U/mL, Thermo Fisher Scientific, Waltham, Mass., USA) and incubated with head-overhead rotation for 30 minutes at 4° C. After 30 minutes, the lysed cells were centrifuged for 15 minutes at 20,000 rpm and the nanobody was subsequently purified from the supernatant (i.e. periplasmic extract) via IMAC. IMAC was performed with Ni-NTA resin (GE Healthcare, Chicago, Ill., USA) in disposable PD-10 columns. The periplasmic extract was mixed with Ni-NTA resin and incubated for 1 h with head-over-head rotation at 9 rpm prior to addition to an empty column. The Ni-NTA resin was washed four times with 8 mL binding buffer (50 mM Tris pH 8.0, 500 mM NaCl and 10 mM imidazole). Next, the nanobody was eluted in three times 700 μL elution buffer (50 mM Tris pH 8.0, 500 mM NaCl and 500 mM imidazole). The elution was further purified by SEC using an enrich70 10×300 column (BioRad, Hercules, Calif., USA). The SEC fractions were analyzed by SDS-PAGE using 4-20% precast protein gels (BioRad, Hercules, Calif., USA), pre-stained protein ladder (#26619, Thermo Fisher Scientific, Waltham, Mass., USA) and InstantBlue protein staining (expedeon, San Diego, Calif., USA). Pure nanobody fractions were pooled together, concentrated to 175 μM and aliquoted for long term storage at −80° C.

Cell Culture and Seeding Protocol for Pharmacological Characterization

All cell lines utilized in this study were grown at 37° C. and 5% CO2. The HEK293A (RRID: CVCL_6910) and stable Flp-In HEK293 cells were cultured in Dulbecco's Modified Eagle Medium (DMEM, #31966021) supplemented with 10% dialyzed Fetal Bovine Serum (dFBS) and 1% 10,000 units/mL penicillin and 10,000 μg/mL streptomycin mixture (pen/strep). The stable Flp-In CHO cells were cultured in F-12K medium (#21127022) supplemented with 10% dFBS and 1% pen/strep. Additionally, the stable Flp-In HEK293 and Flp-In CHO cell lines were cultured in the presence of respectively 200 and 500 μg/mL hygromycin B. For pharmacological characterization, Flp-In HEK293 HA-hCaSR WT cells were seeded in poly-D-lysine-coated clear 96 well tissue culture plates (Corning, Corning, N.Y., USA) at a density of 50,000 cells/well 18 h prior to assay. Experiments with HA-rCaSR WT and HA-hCaSR S170A required transient transfection of HEK293A cells 24 h prior to assay. DNA construct and Lipofectamine 2000 were diluted in Opti-MEM and mixed together after 5 minutes incubation at room temperature. For HA-rCaSR WT, 30 ng/well rCaSR DNA diluted in 25 μL/well Opti-MEM was mixed with 0.25 μL/well Lipofectamine 2000 in 25 μL/well Opti-MEM. For HA-hCaSR S170A, 40 ng/well rCaSR DNA diluted in 25 μL/well Opti-MEM was mixed with 0.25 μL/well Lipofectamine 2000 in 25 μL/well Opti-MEM. The DNA:Lipofectamine 2000 mixtures were incubated 20 minutes at room temperature after which 50 μL/well was added to a poly-D-lysine-coated clear 96 well tissue culture plate. Next, 100 μL/well of a 400,000 cells/mL HEK293A cell solution prepared in culture media was added and the plate was placed at 37° C. and 5% CO2 (final cell density 40,000 cells/well).

Inositol Monophosphate (IP₁) Accumulation Assay

Inositol monophosphate (IP₁) accumulation was measured using the commercially available HTRF® IP1 accumulation kit (Cisbio Bioassays, Codolet, France) and an EnVision multimode plate reader (PerkinElmer, Waltham, Mass., USA). Unless stated otherwise, the cells were washed once with 100 μL/well assay buffer and stimulated for 30 minutes at 37° C. with 50 μL/well ligand(s) diluted in assay buffer supplemented with 20 mM LiCl. Following stimulation, the cells were washed once with 100 μL/well and lysed for 30 minutes at room temperature with 30 μL/well lysis buffer provided with the kit. The lysed cells were diluted 1:2 with assay buffer after which 10 μL/well was pipetted to a white 384-well OptiPlate (PerkinElmer, Waltham, Mass., USA). Next, 10 μL of a detection solution consisting of 2.5% IP₁-d2 conjugate and 2.5% anti-IP₁ antibody diluted in assay buffer was added to each well. Fluorophore emissions were measured after the plate was stored in the dark for 1 h. The 615/665 nm emission ratios measured upon excitation at 340 nm were converted to IP₁ concentrations with an IP1 standard curve according to the manufacturer's instructions.

Advanced Phospho-ERK1/2 Assay

Phosphorylation of ERK1/2 at T202 and Y204 was measured with the HTRF® Advanced Phospho-ERK1/2 assay (Cisbio Bioassays, Codolet, France). The cells were handled gently throughout the procedure to avoid agonist-independent ERK1/2 phosphorylation. Cell culture media was aspirated and the cells were washed twice with 50 μL/well Dulbecco's Phosphate Buffered Saline (DPBS, Thermo Fisher Scientific, Waltham, Mass., USA). Next, the cells were pre-incubated for 30 minutes at room temperature with 50 μL/well nanobody or allosteric modulator or Gq/11 inhibitor YM-25486090 prepared in assay buffer. After pre-incubation, the liquid was aspirated and the cells were stimulated for 10 minutes at room temperature with 50 μL/well CaCl2 solution prepared in assay buffer supplemented with nanobody, allosteric modulator and/or YM-25486090. The agonist solution was removed and the cells were lysed with 50 μL/well lysis buffer (25% lysis solution and 1% blocking reagent diluted in MilliQ) for 1 h at room temperature while shaking at 450 rpm. Part of the lysate (16 μL/well) was transferred to a white 384-well OptiPlate containing 4 μL/well ERK detection solution (assay buffer supplemented with 2.5% advanced phospho-ERK1/2 d2 antibody and 2.5% advanced phospho-ERK1/2 Eu3+-cryptate antibody) and the plate was incubated for 2 h in the dark. Upon excitation at 340 nm, emission at 615 and 665 nm was measured on an EnVision multimode plate reader (PerkinElmer, Waltham, Mass., USA). The resulting 615/665 FRET ratio is used as a direct measure of ERK1/2 phosphorylation.

Data and Statistical Analysis

The data and statistical analysis comply with the recommendations on experimental design and analysis in pharmacology (Curtis et al., 2018). Data were analysed with GraphPad Prism v. 8.2 (GraphPad Software, San Diego, Calif., USA). To ensure the reliability of single values, initial pharmacological screening was performed in duplicate and further pharmacological characterization in triplicate. Concentration-response curves were fitted according to the four-parameter nonlinear regression equation.

Aspects of the Disclosure

A protein binding agent specifically binding the Calcium-sensing receptor (CaSR), characterized in that the protein binding agent is a non-naturally occurring positive allosteric modulator of CaSR activity. Said protein binding agent, specifically binding the CaSR protein when added extracellularly to CaSR-expressing cells.

Said protein binding agent, which is a peptide, a peptidomimetic, an antibody, an antibody mimetic, a single domain antibody, an immunoglobulin single variable domain (ISVD) or an active antibody fragment.

Said protein binding agent, wherein said binding agent is an ISVD comprising 4 framework regions (FR) and 3 complementarity determining regions (CDR) according to the following formula (1): FR1-CDR1-FR2-CDR2-FR3-CDR3-FR4 (1); and wherein: CDR3 consists of a sequence selected from the group of SEQ ID NO: 24-30.

Said ISVD wherein: CDR1 consists of a sequence selected from the group of SEQ ID NO:11-16, and CDR2 consists of a sequence selected from the group of SEQ ID NO:17-23.

Said ISVD, comprising any of the sequences of SEQ ID NO: 3-10, or a sequence with at least 85% amino acid identity thereof, or a humanized variant thereof.

A multi-specific binding agent, comprising any of said protein binding agents disclosed herein.

A pharmaceutical composition comprising any of said protein binding agents disclosed herein.

Said pharmaceutical composition, further comprising a small molecule compound, wherein said compound is a calcimimetic and/or a positive allosteric modulator of CaSR.

Said protein binding agent, or said pharmaceutical composition as disclosed herein, for use as a medicament.

Said protein binding agent, or pharmaceutical composition as disclosed herein, for use in reducing parathyroid hormone (PTH) secretion when administered to a subject.

Said protein binding agent, or the pharmaceutical composition as disclosed herein, for use in treatment of hypercalcemia disorders, such as secondary and primary hyperparathyroidism.

A complex comprising the CaSR extracellular calcium binding domain, and a protein binding agent as disclosed herein. Said complex, wherein said complex is crystalline.

### SEQUENCE LISTING

SEQ ID NO:1: human CaSR wild-type (hCaSR WT) sequence (common Q1011 variant of Uniprot ID P41180; 1078AA).

SEQ ID NO:2 Rattus norvegicus Extracellular calcium-sensing receptor (rCaSR) (Uniprot ID P48442; 1079AA).

SEQ ID NO:3: CaSR Nb10

SEQ ID NO:4: CaSR Nb37

SEQ ID NO:5: CaSR Nb4

SEQ ID NO:6: CaSR Nb11

SEQ ID NO:7: CaSR Nb36

SEQ ID NO:8: CaSR Nb5

SEQ ID NO:9: CaSR Nb2

SEQ ID NO:10: CaSR Nb15

SEQ ID NO:11: CDR1 CaSR Nb10/Nb37

SEQ ID NO:12: CDR1 CaSR Nb4/Nb11

SEQ ID NO:13: CDR1 CaSR Nb36

SEQ ID NO:14: CDR1 CaSR Nb5

SEQ ID NO:15: CDR1 CaSR Nb2

SEQ ID NO:16: CDR1 CaSR Nb 15

SEQ ID NO:17: CDR2 CaSR Nb10/Nb37

SEQ ID NO:18: CDR2 CaSR Nb4

SEQ ID NO:19: CDR2 CaSR Nb11

SEQ ID NO:20: CDR2 CaSR Nb36

SEQ ID NO:21: CDR2 CaSR Nb5

SEQ ID NO:22: CDR2 CaSR Nb2

SEQ ID NO:23: CDR2 CaSR Nb 15

SEQ ID NO:24: CDR3 CaSR Nb10/37

SEQ ID NO:25: CDR3 CaSR Nb4

SEQ ID NO:26: CDR3 CaSR Nb11

SEQ ID NO:27: CDR3 CaSR Nb36

SEQ ID NO:28: CDR3 CaSR Nb5

SEQ ID NO:29: CDR3 CaSR Nb2

SEQ ID NO:30: CDR3 CaSR Nb 15

SEQ ID NO: 31: CaSR Nb36 humanized variant 1

SEQ ID NO: 32: CaSR Nb4_humanized variant 1

SEQ ID NO: 33: CaSR Nb2_humanized variant 1

SEQ ID NO: 34: CaSR Nb10 humanized variant 1

### REFERENCES

Bai, M., Trivedi, S., and Brown, E. M. (1998). Dimerization of the extracellular calcium-sensing receptor (CaR) on the cell surface of CaR-transfected HEK293 cells. J. Biol. Chem. 273: 23605-23610.
Bates, A., and Power, C. A. (2019). David vs. Goliath: The structure, function, and clinical prospects of antibody fragments. Antibodies 8: 1-31.
Bover, J., Urena, P., Ruiz-Garcia, C., Dasilva, I., Lescano, P., Carpio, J. Del, et al. (2016). Clinical and practical use of calcimimetics in dialysis patients With secondary hyperparathyroidism. Clin. J. Am. Soc. Nephrol. 11: 161-174.
Bräuner-Osborne, H., Jensen, A. A., Sheppard, P. O., O'Hara, P., and Krogsgaard-Larsen, P. (1999). The agonist-binding domain of the CaSR is located at the amino-terminal domain. J. Biol. Chem. 274: 18382-18386.
Brini, M., and Carafoli, E. (2011). The plasma membrane Ca²⁺ ATPase and the plasma membrane sodium calcium exchanger cooperate in the regulation of cell calcium. Cold Spring Harb. Perspect. Biol. 3: 1-15.
Brown, E. M. (2013). Role of the calcium-sensing receptor in extracellular calcium homeostasis. Best Pract. Res. Clin. Endocrinol. Metab. 27: 333-343.
Brown, E. M., Fuleihan, G. E.-H., Chen, C. J., and Kifor, O. (1990). A comparison of the effects of divalent and trivalent cations on parathyroid hormone release, 3′,5′-cyclic-adenosine monophosphate accumulation, and the levels of inositol phosphates in bovine parathyroid cells. Endocrinology 127: 1064-1071.
Brown, E. M., Gamba, G., Riccardi, D., Lombardi, M., Butters, R., Kifor, O., et al. (1993). Cloning and characterization of an extracellular Ca²⁺-sensing receptor from bovine parathyroid. Nature 366: 575-580.
Chavez-Abiega, S., Mos, I., Centeno, P. P., Elajnaf, T., Schlattl, W., Ward, D. T., . . . & Kallay, E. (2020). Sensing Extracellular Calcium—An Insight into the Structure and Function of the Calcium-Sensing Receptor (CaSR). In Calcium Signaling (pp. 1031-1063). Springer, Cham.
Conigrave, A. D., Quinn, S. J., and Brown, E. M. (2000). L-Amino acid sensing by the extracellular Ca²⁺-sensing receptor. 97: 4814-4819.
Curtis, M. J., Alexander, S., Cirino, G., Docherty, J. R., George, C. H., Giembycz, M. A., et al. (2018). Experimental design and analysis and their reporting II: updated and simplified guidance for authors and peer reviewers. Br. J. Pharmacol. 175: 987-993.
Dooley, H., and Flajnik, M. F. (2006). Antibody repertoire development in cartilaginous fish. Dev. Comp. Immunol. 30: 43-56.
Falk-Petersen, C. B., Søgaard, R., Madsen, K. L., Klein, A. B., Frølund, B., and Wellendorph, P. (2017). Development of a robust mammalian cell-based assay for studying recombinant a4@31/36 GABAA receptor subtypes. Basic Clin. Pharmacol. Toxicol. 121: 119-129.
Fukagawa, M., Shimazaki, R., and Akizawa, T. (2018). Head-to-head comparison of the new calcimimetic agent evocalcet with cinacalcet in Japanese hemodialysis patients with secondary hyperparathyroidism. Kidney Int. 94: 818-825.
Geng, Y., Mosyak, L., Kurinov, I., Zuo, H., Sturchler, E., Cheng, T. C., et al. (2016). Structural mechanism of ligand activation in human calcium-sensing receptor. Elife 5: 1-25.
Greenberg, A. S., Avila, D., Hughes, M., Hughes, A., McKinney, E. C., and Flajnik, M. F. (1995). A new antigen receptor gene family that undergoes rearrangement and extensive somatic diversification in sharks. Nature 374: 168-173.
Habibullah et al. (2018). Calcium-sensing receptor autoantibodies in patients with autoimmune polyendocrine syndrome type 1: Epitopes, specificity, functional affinity, IgG subclass, and effects on receptor activity. J. Immunol, 201: 3175-3183.
Hamano, N., Komaba, H., and Fukagawa, M. (2017). Etelcalcetide for the treatment of secondary hyperparathyroidism. Expert Opin. Pharmacother. 18: 529-534.
Hamers-Casterman C, Atarhouch, T., Muyldermans, S., Robinson, G., Hamers, C., Songa, E., et al. (1993). Naturally occurring antibodies devoid of light chains. Nature 363: 446-448.
Handlogten, M. E., Shiraishi, N., Awata, H., Huang, C., and Miller, R. T. (2000). Extracellular Ca²⁺-sensing receptor is a promiscuous divalent cation sensor that responds to lead. Am. J. Physiol.—Ren. Physiol. 279: 1083-1091.
Hannan, F. M., Kallay, E., Chang, W., Brandi, M., and Thakker, R. (2018). The calcium-sensing receptor in physiology and in calcitropic and non-calcitropic diseases. Nat. Rev. Endocrinol. 15: 33-51.
Heukers, R., Groof, T. W. M. De, and Smit, M. J. (2019). Nanobodies detecting and modulating GPCRs outside in and inside out. Curr. Opin. Cell Biol. 57: 115-122.
Hofer, A. M., and Brown, E. M. (2003). Extracellular calcium sensing and signalling. Nat. Rev. Mol. Cell Biol. 4: 530-538.
Jacobsen, S. E., Gether, U., and Bräuner-Osborne, H. (2017). Investigating the molecular mechanism of positive and negative allosteric modulators in the calcium-sensing receptor dimer. Sci. Rep. 7: 1-14.
Johansson, H., Cailly, T., Thomsen, A. R. B., Brauner-Osborne, H., and Pedersen, D. S. (2013). Synthesis of the calcilytic ligand NPS 2143. Beilstein J. Org. Chem. 9: 1383-1387.
Kawata, T., Tokunaga, S., Murai, M., Masuda, N., Haruyama, W., Shoukei, Y., et al. (2018). A novel calcimimetic agent, evocalcet (MT-4580/KHK7580), suppresses the parathyroid cell function with little effect on the gastrointestinal tract or CYP isozymes in vivo and in vitro. PLoS One 13: 1-14.
Koehl, A., Hu, H., Feng, D., Sun, B., Zhang, Y., Robertson, M. J., et al. (2019). Structural insights into the activation of metabotropic glutamate receptors. Nature 566: 79-84.
Li et al. (1996). Autoantibodies to the extracellular domain of the calcium sensing receptor in patients with acquired hypoparathyroidism. J Clin Invest. 97(4): 910-914.
Meier, C., Ristic, Z., Klauser, S., and Verrey, F. (2002). Activation of system L heterodimeric amino acid exchangers by intracellular substrates. EMBO J. 21: 580-589.
Morrison, C. (2019). Nanobody approval gives domain antibodies a boost. Nat. Rev. Drug Discov. 18: 485-487.
Mos, I., Jacobsen, S. E., Foster, S., and Brauner-Osborne, H. (2019). Calcium-sensing receptor internalization is @3-arrestin-dependent and modulated by allosteric ligands. Mol. Pharmacol. Epub ahead of print.
Pardon, E., Laeremans, T., Triest, S., Rasmussen, S., Wohlkonig, A., Ruf, A., et al. (2014). A general protocol for the generation of Nanobodies for structural biology. Nat. Protoc. 9: 674-693.
Patel, J., and Bridgeman, M. B. (2018). Etelcalcetide (Parsabiv) for secondary hyperparathyroidism in adults with chronic kidney disease on hemodialysis. P T 43: 396-399.
Poon, G. (2005). Cinacalcet hydrochloride (Sensipar). Proc (Bayl Univ Med Cent). 18: 181-184.
Vahe, C., Benomar, K., Espiard, S., Coppin, L., Jannin, A., Odou, M. F., et al. (2017). Diseases associated with calcium-sensing receptor. Orphanet J. Rare Dis. 12: 1-9.
Zhang, C., Zhang, T., Zou, J., Miller, C. L., Gorkhali, R., Yang, J. Y., et al. (2016). Structural basis for regulation of human calcium-sensing receptor by magnesium ions and an unexpected tryptophan derivative co-agonist. Sci. Adv. 2: 1-9.
1. Kobilka, B. K. (2007) G protein coupled receptor structure and activation. Biochim Biophys Acta. 1768, 794-807
23. Fitzpatrick, L. A., Brandi, M. L., and Aurbach, G. D. (1986) Control of PTH secretion is mediated through calcium channels and is blocked by pertussis toxin treatment of parathyroid cells. Biochem Biophys Res Commun. 138, 960-965
24. Brown, E. M., Gamba, G., Riccardi, D., Lombardi, M., Butters, R., Kifor, O., Sun, A., Hediger, M. A., Lytton, J., and Hebert, S. C. (1993) Cloning and characterization of an extracellular Ca(2+)-sensing receptor from bovine parathyroid. Nature. 366, 575-580
28. Brown, E. M., and MacLeod, R. J. (2001) Extracellular calcium sensing and extracellular calcium signaling. Physiol Rev. 81, 239-297
29. Quinn, S. J., Bai, M., and Brown, E. M. (2004) pH Sensing by the calcium-sensing receptor. J. Biol. Chem. 279, 37241-37249
30. Broadhead, G. K., Mun, H. C., Avlani, V. A., Jourdon, O., Church, W. B., Christopoulos, A., Delbridge, L., and Conigrave, A. D. (2011) Allosteric modulation of the calciumsensing receptor by γ-glutamyl peptides: Inhibition of PTH secretion, suppression of intracellular cAMP levels, and a common mechanism of action with L-amino acids. J. Biol. Chem. 286, 8786-8797
40. Kunishima, N., Shimada, Y., Tsuji, Y., Sato, T., Yamamoto, M., Kumasaka, T., Nakanishi, S., Jingami, H., and Morikawa, K. (2000) Structural basis of glutamate recognition by a dimeric metabotropic glutamate receptor. Nature. 407, 971-977
42. Muto, T., Tsuchiya, D., Morikawa, K., and Jingami, H. (2007) Structures of the extracellular regions of the group II/III metabotropic glutamate receptors. Proc. Natl. Acad. Sci. 104, 3759-3764
43. Tsuchiya, D., Kunishima, N., Kamiya, N., Jingami, H., and Morikawa, K. (2002) Structural views of the ligand-binding cores of a metabotropic glutamate receptor complexed with an antagonist and both glutamate and Gd3+. Proc. Natl. Acad. Sci. U. S. 99, 2660-2665
46. Geng, Y., Mosyak, L., Kurinov, I., Zuo, H., Sturchler, E., Cheng, T. C., Subramanyam, P., Brown, A. P., Brennan, S. C., Mun, H. C., Bush, M., Chen, Y., Nguyen, T. X., Cao, B., Chang, D. D., Quick, M., Conigrave, A. D., Colecraft, H. M., McDonald, P., and Fan, Q. R. (2016) Structural mechanism of ligand activation in human calcium-sensing receptor. Elife. 5, 1-25
47. Zhang, C., Zhang, T., Zou, J., Miller, C. L., Gorkhali, R., Yang, J. Y., Schilmiller, A., Wang, S., Huang, K., Brown, E. M., Moremen, K. W., Hu, J., and Yang, J. J. (2016) Structural basis for regulation of human calcium-sensing receptor by magnesium ions and an unexpected tryptophan derivative co-agonist. Sci. Adv. 2, 1-9
51. Huang, Y., Zhou, Y., Castiblanco, A., Yang, W., Brown, E. M., and Yang, J. J. (2009) Multiple Ca(2+)-binding sites in the extracellular domain of the Ca(2+)-sensing receptor corresponding to cooperative Ca(2+) response. Biochemistry. 48, 388-398
52. Bai, M., Quinn, S., Trivedi, S., Kifor, O., Pearce, S. H., Pollak, M. R., Krapcho, K., Hebert, S. C., and Brown, E. M. (1996) Expression and characterization of inactivating and activating mutations in the human Ca²⁺(o)-sensing receptor. J. Biol. Chem. 271, 19537-19545
53. Zhang, C., Zhuo, Y., Moniz, H. A., Wang, S., Moremen, K. W., Prestegard, J. H., Brown, E. M., and Yang, J. J. (2014) Direct determination of multiple ligand interactions with the extracellular domain of the calcium-sensing receptor. J. Biol. Chem. 289, 33529-33542
54. Brown, E. M. (2013) Role of the calcium-sensing receptor in extracellular calcium homeostasis. Best Pract. Res. Clin. Endocrinol. Metab. 27, 333-343
55. Zhang, C., Miller, C. L., Brown, E. M., and Yang, J. J. (2015) The calcium sensing receptor: from calcium sensing to signaling. Sci. China Life Sci. 58, 14-27
56. Brown, E. M. (2007) Clinical lessons from the calcium-sensing receptor. Nat. Clin. Pract. Endocrinol. Metab. 3, 122-133
57. Hendy, G. N., Guarnieri, V., and Canaff, L. (2009) Calcium-sensing receptor and associated diseases, 1st Ed., Elsevier Inc., 10.1016/S1877-1173(09)89003-0
58. Nemeth, E. F., Van Wagenen, B. C., and Balandrin, M. F. (2018) Discovery and development of calcimimetic and calcilytic compounds. Prog. Med. Chem. 57, 1-86
59. Nemeth, E. F., Steffey, M. E., Hammerland, L. G., Hung, B. C., Van Wagenen, B. C., DelMar, E. G., and Balandrin, M. F. (1998) Calcimimetics with potent and selective activity on the parathyroid calcium receptor. Proc. Natl. Acad. Sci. U.S.A. 95, 4040-4045
60. Zanger, U. M., Fischer, J., Raimundo, S., Stüven, T., Evert, B. O., Schwab, M., and Eichelbaum, M. (2001) Comprehensive analysis of the genetic factors determining expression and function of hepatic CYP2D6. Pharmacogenetics. 11, 573-585
64. Block, G. A., Zaun, D., Smits, G., Persky, M., Brillhart, S., Nieman, K., Liu, J., and St Peter, W. L. (2010) Cinacalcet hydrochloride treatment significantly improves all-cause and cardiovascular survival in a large cohort of hemodialysis patients. Kidney Int. 78, 578-589
66. Silverberg, S. J., Rubin, M. R., Faiman, C., Peacock, M., Shoback, D. M., Smallridge, R. C., Schwanauer, L. E., Olson, K. A., Klassen, P., and Bilezikian, J. P. (2007) Cinacalcet hydrochloride reduces the serum calcium concentration in inoperable parathyroid carcinoma. J. Clin. Endocrinol. Metab. 92, 3803-3808
67. Bover, J., Ureña, P., Ruiz-García, C., DaSilva, I., Lescano, P., del Carpio, J., Ballarín, J., and Cozzolino, M. (2016) Clinical and practical use of calcimimetics in dialysis patients with secondary hyperparathyroidism. Clin J Am Soc Nephrol. 11, 161-174
71. Fukagawa, M., Shimazaki, R., and Akizawa, T. (2018) Head-to-head comparison of the new calcimimetic agent evocalcet with cinacalcet in Japanese hemodialysis patients with secondary hyperparathyroidism. Kidney Int. 94, 818-825
72. Hamano, N., Komaba, H., and Fukagawa, M. (2017) Etelcalcetide for the treatment of secondary hyperparathyroidism. Expert Opin. Pharmacother. 18, 529-534
73. Block, G. A., Bushinsky, D. A., Cheng, S., Cunningham, J., Dehmel, B., Drueke, T. B., Ketteler, M., Kewalramani, R., Martin, K. J., Moe, S. M., Patel, U. D., Silver, J., Sun, Y., Wang, H., and Chertow, G. M. (2017) Effect of etelcalcetide vs cinacalcet on serum parathyroid hormone in patients receiving hemodialysis with secondary hyperparathyroidism: A randomized clinical trial. JAMA—J. Am. Med. Assoc. 317, 156-164
74. Parfrey, P. S. (2018) Evocalcet in the management of secondary hyperparathyroidism in dialysis patients. Kidney Int. 94, 661-662
75. Patel, J., and Bridgeman, M. B. (2018) Etelcalcetide (Parsabiv) for secondary hyperparathyroidism in adults with chronic kidney disease on hemodialysis. P T. 43, 396-399
83. Casalà, C., Gil-Guiñón, E., Ordóñez, J. L., Miguel-Queralt, S., Rodríguez, E., Galván, P., Lavarino, C., Munell, F., de Alava, E., Mora, J., and De Torres, C. (2013) The calciumsensing receptor is silenced by genetic and epigenetic mechanisms in unfavourable neuroblastomas and its reactivation induces ERK1/2-dependent apoptosis. Carcinogenesis. 34, 268-276
84. Rodriguez-Hernandez, C. J., Mateo-Lozano, S., Garcia, M., Casalà, C., Briansó, F., Castrejón, N., Rodríguez, E., Suñol, M., Carcaboso, A. M., Lavarino, C., Mora, J., and De Torres, C. (2016) Cinacalcet inhibits neuroblastoma tumor growth and upregulates cancertestis antigens. Oncotarget. 7, 16112-16129
85. Bräuner-Osborne, H.; Jensen, A. A.; Krogsgaard-Larsen, P., Interaction of CPCCOEt with a chimeric mGlu_{1b} and calcium sensing receptor. Neuroreport 1999, 10, 3923-3925.
86. Bräuner-Osborne, H.; Jensen, A. A.; Sheppard, P. O.; O'Hara, P.; Krogsgaard-Larsen, P., The agonist-binding domain of the calcium-sensing receptor is located at the amino-terminal domain. J. Biol. Chem. 1999, 274, 18382-18386.
87. Jørgensen, S.; Have, C. T.; Underwood, C. R.; Johansen, L. D.; Wellendorph, P.; Gjesing, A. P.; Jørgensen, C. V.; Quan, S.; Rui, G.; Inoue, A.; Linneberg, A.; Grarup, N.; Jun, W.; Pedersen, O.; Hansen, T.; Brauner-Osborne, H., Genetic variations in the human G protein-coupled receptor class C, group 6, member A (GPRC6A) control cell surface expression and function. J. Biol. Chem. 2017, 292, 1524-1534.

1. An antibody or active antibody fragment specifically binding the extracellular domain of the Calcium-sensing receptor (CaSR), wherein the antibody or active antibody fragment is a non-naturally occurring positive allosteric modulator of CaSR activity.

2.-15. (canceled)

16. A polypeptide comprising a sequence selected from the group of SEQ ID NOs: 24-30.

17. The polypeptide of claim 16, wherein the polypeptide further comprises a sequence selected from the group of SEQ ID NOs:11-16, and a sequence selected from the group of SEQ ID NOs:17-23.

18. The polypeptide of claim 17, wherein the polypeptide comprises one or more of the sequences of SEQ ID NOs: 3-10, or a sequence with at least 85% amino acid identity thereof.

19. The polypeptide of claim 18, wherein the polypeptide comprises one or more of the sequences of SEQ ID NOs: 31-34.

20. The polypeptide of claim 16, wherein the polypeptide is an antibody or active antibody fragment specifically binding the extracellular domain of the Calcium-sensing receptor (CaSR), wherein the antibody or active antibody fragment is a non-naturally occurring positive allosteric modulator of CaSR activity.

21. The polypeptide of claim 20, wherein the antibody or active antibody comprises an immunoglobulin single variable domain (ISVD), wherein CDR3 consists of a sequence selected from the group of SEQ ID NO: 24-30.

22. The polypeptide of claim 21, wherein CDR1 of the ISVD consists of a sequence selected from the group of SEQ ID NO:11-16, and CDR2 of the ISVD consists of a sequence selected from the group of SEQ ID NO:17-23.

23. The polypeptide of claim 16, wherein the polypeptide is comprised in a multivalent or multi-specific binding agent.

24. The polypeptide of claim 16, wherein the polypeptide is comprised in a pharmaceutical composition.

25. The polypeptide of claim 24, wherein the pharmaceutical composition further comprises a small molecule compound, wherein said compound is a calcimimetic and/or a positive allosteric modulator of Calcium-sensing receptor (CaSR).

26. The polypeptide of claim 25, wherein the polypeptide comprises an immunoglobulin single variable domain (ISVD) with CDR1 selected from SEQ ID NO:11, 12 or 15; CDR2 from SEQ ID NO:17, 18, or 22, and CDR3 from SEQ ID NO:24, 25, or 29.

27. The polypeptide of claim 25, wherein the polypeptide comprises an immunoglobulin single variable domain (ISVD), the ISVD comprising a sequence selected from the group of SEQ ID NO: 3, 5, 9, 32, 33 or 34, or a humanized variant thereof.

28. A method of administering a polypeptide to a subject, the method comprising: administering to the subject the polypeptide of claim 16.

29. The method of claim 28, where the administration of the polypeptide reduces parathyroid hormone (PTH) secretion in the subject.

30. The method of claim 28, wherein the subject suffers from a hypercalcemia disorder and wherein the administration of the polypeptide treats the hypercalcemia disorder in the subject.

31. The method of claim 30, wherein the hypercalcemia disorder is secondary or primary hyperparathyroidism.


SEQUENCE LISTING (34 sequences)

SEQ ID NO: 1 - protein, 1075 aa, Homo sapiens
MAFYSCCWVLLALTWHTSAYGPDQRAQKKGDIILGGLFPIHFGVAAKDQDLKSRPESVECIRYNFRGFRWLQAMIFAIEEINSSPALLPNLTLGYRIFDTCNTVSKALEATLSFVAQNKIDSLNLDEFCNCSEHIPSTIAVVGATGSGVSTAVANLLGLFYIPQVSYASSSRLLSNKNQFKSFLRTIPNDEHQATAMADIIEYFRWNWVGTIAADDDYGRPGIEKFREEAEERDICIDFSELISQYSDEEEIQHVVEVIQNSTAKVIVVFSSGPDLEPLIKEIVRRNITGKIWLASEAWASSSLIAMPQYFHVVGGTIGFALKAGQIPGFREFLKKVHPRKSVHNGFAKEFWEETFNCHLQEGAKGPLPVDTFLRGHEESGDRFSNSSTAFRPLCTGDENISSVETPYIDYTHLRISYNVYLAVYSIAHALQDIYTCLPGRGLFTNGSCADIKKVEAWQVLKHLRHLNFTNNMGEQVTFDECGDLVGNYSIINWHLSPEDGSIVFKEVGYYNVYAKKGERLFINEEKILWSGFSREVPFSNCSRDCLAGTRKGIIEGEPTCCFECVECPDGEYSDETDASACNKCPDDFWSNENHTSCIAKEIEFLSWTEPFGIALTLFAVLGIFLTAFVLGVFIKFRNTPIKATNRELSYLLLFSLLCCFSSSLFFIGEPQDWTCRLRQPAFGISFVLCISCILVKTNRVLLVFEAKIPTSFHRKWWGLNLQFLLVFLCTFMQIVICVIWLYTAPPSSYRNQELEDEIIFITCHEGSLMALGFLIGYTCLLAAICFFFAFKSRKLPENFNEAKFITFSLIFFIVWISFIPAYASTYGKFVSAVEVIAILAASFGLLACIFFNKIYIILFKPSRNTIEEVRCSTAAHAFKVAARATLRRSNVSRKSSSLGGSTGSTPSSSISSKSNSEDPFPQPERQKQQQPLALTQQEQQQQPLTLPQQQRSQQQPRCKQKVIFGSGTVTFSLSFDEPQKNAMAHRNSTHQNSLEAQKSSDTLTRHQPLLPLQCGETDLDLTVQETGLQGPVGGDQRPEVEDPEELSPALVVSSSQSFVISGGGSTVTENVVNS

SEQ ID NO: 2 - protein, 1079 aa, Rattus norvegicus
MASYSCCLALLALAWHSSAYGPDQRAQKKGDIILGGLFPIHFGVAAKDQDLKSRPESVECIRYNFRGFRWLQAMIFAIEEINSSPSLLPNMTLGYRIFDTCNTVSKALEATLSFVAQNKIDSLNLDEFCNCSEHIPSTIAVVGATGSGVSTAVANLLGLFYIPQVSYASSSRLLSNKNQYKSFLRTIPNDEHQATAMADIIEYFRWNWVGTIAADDDYGRPGIEKFREEAEERDICIDFSELISQYSDEEEIQQVVEVIQNSTAKVIVVFSSGPDLEPLIKEIVRRNITGRIWLASEAWASSSLIAMPEYFHVVGGTIGFGLKAGQIPGFREFLQKVHPRKSVHNGFAKEFWEETFNCHLQEGAKGPLPVDTFVRSHEEGGNRLLNSSTAFRPLCTGDENINSVETPYMDYEHLRISYNVYLAVYSIAHALQDIYTCLPGRGLFTNGSCADIKKVEAWQVLKHLRHLNFTNNMGEQVTFDECGDLVGNYSIINWHLSPEDGSIVFKEVGYYNVYAKKGERLFINEEKILWSGFSREVPFSNCSRDCQAGTRKGIIEGEPTCCFECVECPDGEYSGETDASACDKCPDDFWSNENHTSCIAKEIEFLAWTEPFGIALTLFAVLGIFLTAFVLGVFIKFRNTPIVKATNRELSYLLLFSLLCCFSSSLFFIGEPQDWTCRLRQPAFGISFVLCISCILVKTNRVLLVFEAKIPTSFHRKWWGLNLQFLLVFLCTFMQILICIIWLYTAPPSSYRNHELEDEIIFITCHEGSLMALGSLIGYTCLLAAICFFFAFKSRKLPENFNEAKFITFSMLIFFIVWISFIPAYASTYGKFVSAVEVIAILAASFGLLACIFFNKVYIILFKPSRNTIEEVRSSTAAHAFKVAARATLRRPNISRKRSSSLGGSTGSIPSSSISSKSNSEDRFPQPERQKQQQPLSLTQQEQQQQPLTLHPQQQQQPQQPRCKQKVIFGSGTVTFSLSFDEPQKNAMAHRNSMRQNSLEAQRSNDTLGRHQALLPLQCADADSEMTIQETGLQGPMVGDHQPEMESSDEMSPALVMSTSRSFVISGGGSSVTENVLHS

SEQ ID NO: 3 - protein, 128 aa, Artificial Sequence
  CaSR Nb10
QVQLVESGGGLVQPGDSLRVSCAASGGSFRSYAMAWFRQAPGKEREFVAAMSGTYGSTIYSDSMKGRFTISRDNAKKMVYLRMNSLRPEDTAVYYCAGYYSGAYYGTPKTDYTEYDSWGQGTQVTVSS

SEQ ID NO: 4 - protein, 128 aa, Artificial Sequence
  CaSR Nb37
QVQLVESGGGLVQPGDSLRVSCAASGGSFRSYAMAWFRRAPGKEREFVAAMSGTYGSTIYADSMKGRFTISRDNAKKMVYLRMNSLRAEDTAVYYCAGYYSGAYYGTPKTDYTEYDSWGQGTQVTVSS

SEQ ID NO: 5 - protein, 128 aa, Artificial Sequence
  CaSR Nb4
QVQLVESGGGLVQAGGSLRLSCAASGRTFSTYRMGWFRQGLGKEREFVAAITANGVRTFYTDSVKGRFTISRDNAKNTLYLQMNSLQPDDTAVYLCAARRKGIFTVQQAQDITDWDYWGQGTQVTVSS

SEQ ID NO: 6 - protein, 128 aa, Artificial Sequence
  CaSR Nb11
QVQLVESGGGLVQAGGSLRLSCAASGRTFSTYRMGWFRQGPGKEREFVAAITATGVKTYYTDSVKGRFTISRDNAKNTVYLQMNSVQPEDTAVYSCAARIKAIFTAQQAATITDYDYWGQGTQVTVSS

SEQ ID NO: 7 - protein, 128 aa, Artificial Sequence
  CaSR Nb36
QVQLVESGGGLVQAGESLRLSCAASGRTFSSYRMGWFRQGPGKEREFVAAITATGVRTYYADSVKGRFTISRDNAKNTVYLQMNSLQPDDTAVYLCAARRKAIFSVQQALTITDYDDWGQGTQVTVSS

SEQ ID NO: 8 - protein, 124 aa, Artificial Sequence
  CaSR Nb5
QVQLVESGGRLVQDGGSLRLSCAASGLTFSDYSMGWFRQVPGKEREFVAVIRRSNDSTYYADFVKGRFTISRDNAKNTVYLQMNSLQPEDTAVYSCAADRGGVGLTSAQTYAYWGQGTQVTVSS

SEQ ID NO: 9 - protein, 125 aa, Artificial Sequence
  CaSR Nb2
QVQLVESGGGLVQAGSSLRLSCAASGRTFSNYRMAWFRQAPGKEREFVAVISRGGGGADYADSVKGRFTISRDNVKNTVSLQMNSLKPEDTAVYFCAARISGFSYYTSVDRYDYWGQGTQVTVSS

SEQ ID NO: 10 - protein, 125 aa, Artificial Sequence
  CaSR Nb15
QVQLVESGGGLVQAGGSLRLSCAASGRTFNNYHMAWFRQAPGKEREFAAAISRRAGTTYYADSVKGRFTISRDNAKNTVYLQMNSLKPEDTAVYYCAADPDTYGVSYLARKYDYWGQGTQVTVSS

SEQ ID NO: 11 - protein, 8 aa, Artificial Sequence
  CDR1 CaSR Nb10/Nb37
GGSFRSYA

SEQ ID NO: 12 - protein, 8 aa, Artificial Sequence
  CDR1 CaSR Nb4/Nb11
GRTFSTYR

SEQ ID NO: 13 - protein, 8 aa, Artificial Sequence
  CDR1 CaSR Nb36
GRTFSSYR

SEQ ID NO: 14 - protein, 8 aa, Artificial Sequence
  CDR1 CaSR Nb5
GLTFSDYS

SEQ ID NO: 15 - protein, 8 aa, Artificial Sequence
  CDR1 CaSR Nb2
GRTFSNYR

SEQ ID NO: 16 - protein, 8 aa, Artificial Sequence
  CDR1 CaSR Nb 15
GRTFNNYH

SEQ ID NO: 17 - protein, 8 aa, Artificial Sequence
  CDR2 CaSR Nb10/Nb37
MSGTYGST

SEQ ID NO: 18 - protein, 8 aa, Artificial Sequence
  CDR2 CaSR Nb4
ITANGVRT

SEQ ID NO: 19 - protein, 8 aa, Artificial Sequence
  CDR2 CaSR Nb11
ITATGVKT

SEQ ID NO: 20 - protein, 8 aa, Artificial Sequence
  CDR2 CaSR Nb36
ITATGVRT

SEQ ID NO: 21 - protein, 8 aa, Artificial Sequence
  CDR2 CaSR Nb5
IRRSNDST

SEQ ID NO: 22 - protein, 8 aa, Artificial Sequence
  CDR2 CaSR Nb2
ISRGGGGA

SEQ ID NO: 23 - protein, 8 aa, Artificial Sequence
  CDR2 CaSR Nb 15
ISRRAGTT

SEQ ID NO: 24 - protein, 21 aa, Artificial Sequence
  CDR3 CaSR Nb10/37
AGYYSGAYYGTPKTDYTEYDS

SEQ ID NO: 25 - protein, 18 aa, Artificial Sequence
  CDR3 CaSR Nb4
AARRKGIFTVQQAITDDY

SEQ ID NO: 26 - protein, 18 aa, Artificial Sequence
  CDR3 CaSR Nb11
AARKAIFTQQATITDYDY

SEQ ID NO: 27 - protein, 19 aa, Artificial Sequence
  CDR3 CaSR Nb36
AARRKAIFVQQATITDYDD

SEQ ID NO: 28 - protein, 17 aa, Artificial Sequence
  CDR3 CaSR Nb5
AADRGGVGLTSAQTYAY

SEQ ID NO: 29 - protein, 18 aa, Artificial Sequence
  CDR3 CaSR Nb2
AARISGFSYYTSVDRYDY

SEQ ID NO: 30 - protein, 18 aa, Artificial Sequence
  CDR3 CaSR Nb 15
AADPDTYGVSYLARKYDY

SEQ ID NO: 31 - protein, 128 aa, Artificial Sequence
  CaSR Nb36 humanized variant 1
EVQLVESGGGLVQPGGSLRLSCAASGRTFSSYRMGWFRQAPGKEREFVAAITATGVRTYYADSVKGRFTISRDNAKNTVYLQMNSLRPEDTAVYYCAARRKAIFSVQQALTITDYDDWGQGTLVTVSS

SEQ ID NO: 32 - protein, 128 aa, Artificial Sequence
  CaSR Nb4_humanized variant 1
EVQLVESGGGLVQPGGSLRLSCAASGRTFSTYRMGWFRQAPGKEREFVAAITANGVRTFYADSVKGRFTISRDNAKNTLYLQMNSLRPEDTAVYYCAARRKGIFTVQQAQDITDWDYWGQGTLVTVSS

SEQ ID NO: 33 - protein, 125 aa, Artificial Sequence
  CaSR Nb2_humanized variant 1
EVQLVESGGGLVQPGGSLRLSCAASGRTFSNYRMAWFRQAPGKEREFVAVISRGGGGADYADSVKGRFTISRDNAKNTVYLQMNSLRPEDTAVYYCAARISGFSYYTSVDRYDYWGQGTLVTVSS

SEQ ID NO: 34 - protein, 128 aa, Artificial Sequence
  CaSR Nb10 humanized variant 1
EVQLVESGGGLVQPGGSLRLSCAASGGSFRSYAMAWFRQAPGKEREFVAAMSGTYGSTIYADSVKGRFTISRDNAKNTVYLQMNSLRPEDTAVYYCAGYYSGAYYGTPKTDYTEYDSWGQGTLVTVSS